SEQUENCE LISTING (21 sequences)

SEQ ID NO: 1 - protein, 360 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 2 - protein, 352 aa, Homo sapiens [sequence omitted]

SEQ ID NO: 3 - protein, 355 aa, Homo sapiens
METPNTTEDYDTTTEFDYGDATPCQKVNERAFGAQLLPPLYSLVFVIGLVGNILVVLVLVQYKRLKNMTSIYLLNLAISDLLFLFTLPFWIDYKLKDDWVFGDAMCKILSGFYYTGLYSEIFFIILLTIDRYLAIVHAVFALRARTVTFGVITSIIIWALAILASMPGLYFSKTQWEFTHHTCSLHFPHESLREWKLFQALKLNLFGLVLPLLVMIICYTGIIKILLRRPNEKKSKAVRLIFVIMIIFFLFWTPYNLTILISVFQDFLFTHECEQSRHLDLAVQVTEVIAYTHCCVNPVIYAFVGERFRKYLRQLFHRRVAVHLVKWLPFLSVDRLERVSSTSPSTGEHELSAGF

SEQ ID NO: 4 - protein, 356 aa, Homo sapiens
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 5 - protein, 362 aa, Homo sapiens
MDLHLFDYSEPGNFSDISWPCNSSDCIVVDTVMCPNMPNKSVLLYTLSFIYIFIFVIGMIANSVVVWVNIQAKTTGYDTHCYILNLAIADLWVVLTIPVWVVSLVQHNQWPMGELTCKVTHLIFSINLFGSIFFLTCMSVDRYLSITYFTNTPSSRKKMVRRVVCILVWLLAFCVSLPDTYYLKTVTSASNNETYCRSFYPEHSIKEWLIGMELVSVVLGFAVPFSIIAVFYFLLARAISASSDQEKHSSRKIIFSYVVVFLVCWLPYHVAVLLDIFSILHYIPFTCRLEHALFTALHVTQCLSLVHCCVNPVLYSFINRNYRYELMKAFIFKYSAKTGLTKLIDASRVSETEYSALEQSTK

SEQ ID NO: 6 - protein, 309 aa, Homo sapiens
MNGTYNTCGSSDLTWPPAIKLGFYAYLGVLLVLGLLLNSLALWVFCCRMQQWTETRIYMTNLAVADLCLLCTLPFVLHSLRDTSDTPLCQLSQGIYLTNRYMSISLVTAIAVDRYVAVRHPLRARGLRSPRQAAAVCAVLWVLVIGSLVARWLLGIQEGGFCFRSTRHNFNSMAFPLLGFYLPLAVVVFCSLKVVTALAQRPPTDVGQAEATRKAARMVWANLLVFVVCFLPLHVGLTVRLAVGWNACALLETIRRALYITSKLSDANCCLDAICYYYMAKEFQEASALAVAPSAKAHKSQDSLCVTLA

SEQ ID NO: 7 - protein, 348 aa, Artificial Sequence
  Consensus sequence
MPTNEDYXEXTFDXDYEPCQKSDVKQXGALQLLPPLYSLVFVXGXVGNXLVVLVLIXCKXLKXMTDIYLLNLAIXCLLFLXTLPFWAHSAXANWXFGNAMCKLLXGIYXIXLXSSIFFIILLTIDRYLAIVHAVFALXARTVTFGVVXSVIXWXLAXXASLPGIIFXKTQKEDXHYTCSXXFPHXEWKNFQTLKXXXLGLVLPLLVMVICYSGILKTLLRXXNEKKRHKAVRLIFTIMIVFFLXWLPYNIXILLXTFQEFFGXXNCEXXNTLDQALQVTETLXMTHCCXNPIIYAFVGEKFRXYLXVFFHKHIAXRFCKXPIFSXEAXEGVSSXXTPSTGEQEXSAGL

SEQ ID NO: 8 - protein, 323 aa, Felis catus
MSRGGGDGSAQREKPQGRGVGSLPHCARRAGLSGRKTRRLGVSSEWGGRPRRPLEQGCPCAQSHRKSPVWVRTAGPEEPLCPRTSRRTMNGTCHSSELTWPYWVKNIVDAYVGLLLALGLLLNGLALWVFCCRVRRWTETHIYMANLAVADLCLLCALPFFLYSLKQRGVREGGFCFLSASRHSSNTTAFSLLGFYLPLAVLLFCSLRVVAALGQGPAAHTDQAEATRRATRMVWANLVVFVACFLPLHVVLAVHVAASRSAPAPALCYALYVTSKLSDANCCLDAICYYFMAKEFQEEAAALASMPTAKAHRSRDSFSVTLA

SEQ ID NO: 9 - protein, 379 aa, Bos taurus
MPTSCSIASTIFSQTPTIPRGEGPRPQGRRSPCLQSRIWTPTRHRLPHRLLGRCEAGEPFGRTCRGVSSSGGNQRVSPGIMNSSNCSSWDANPVYYTYMGGLLALGLLLNGLALWVLCWRLPRWTETRIYMANLAVADLCLLCALPSFLYFQKQTSKDTPLCQISQAVYLLNRYMSISLVTAIAVDRYVAVRHPLRARRLRSPGRAAAVCTALWAVVLGSLVLRWFLDVQDGGFCFAVRSGRSTYTGVFSLLGFYLPLAVLVFCSLQVVTALTQRPEANPGQAEATQKASRMVLANLAVFVVCFLPFHMVLTMRVALGLQTCAIKVAMQITSRLSDANCCLDAICYYFMAKEFQEASVSTTSPRAKAHKSKDSVTMTLT

SEQ ID NO: 10 - protein, 340 aa, Homo sapiens
MLSGSRAVPTPHRGSEELLKYMLHSPCVSLTMNGTYNTCGSSDLTWPPAIKLGFYAYLGVLLVLGLLLNSLALWVFCCRMQQWTETRIYMTNLAVADLCLLCTLPFVLHSLRDTSDTPLCQLSQGIYLTNRYMSISLVTAIAVDRYVAVRHPLRARGLRSPRQAAAVCAVLWVLVIGSLVARWLLGIQEGGFCFRSTRHNFNSMAFPLLGFYLPLAVVVFCSLKVVTALAQRPPTDVGQAEATRKAARMVWANLLVFVVCFLPLHVGLTVRLAVGWNACALLETIRRALYITSKLSDANCCLDAICYYYMAKEFQEASALAVAPSAKAHKSQDSLCVTLA

SEQ ID NO: 11 - protein, 300 aa, Pan troglodytes
MLCLRCFVFVDMGLGLTSSHIPAQRWAEWGQCLAPPARSLLTSGSLCCPRTMNGTYNTCGSSDLTWPPHSLRDTSDTPLCQLSQGIYLTNRYMSISLVTAIAVDRYVAVRHPLRARGLRSPRQAAAVCAVLWVLVIGSLVARWFLGMQEGGFCFRSTRHNFNSMAFPLLGFYLPLAVVVFCSLKVVTALAQRPPTDVGQAEATRKAARMVWANLVVFVVCFLPLHVGLTVRLAVGWNACALLETVRRALYITSKLSDANCCLDAICYYYMAKEFQEASALAVAPSAKAHKSQDSLCVTLA

SEQ ID NO: 12 - protein, 309 aa, Macaca mulatta
MNGTYNTCGSSDLTWPPTIKLGFYAYLGILLVLGLLLNSLALWVFCCRMQRWTETRIYMTNLAVADLCLLCALPFVLHSLQDTSDTPLCQLSQGIYLTNRYMSISLVTAIAVDRYVAVRHPLRARGLRSPRQAAAVCAVLWMLVIGSLVARWFLGMQEGGFCFRSTRHNFSSMAFPLLGFYLPLAVVVFCSLKVVTALAQRPPIDVGQAEASHKAARMVWANLVVFVVCFLPLHVGLTVRLTVGWNACAFLETLRRTLFITSKLSDANCCLDAICYYYMAKEFQEASALAVVPSAKAHKSQASLCVTLA

SEQ ID NO: 13 - protein, 306 aa, Rattus norvegicus
MNNTNCSILPWPAAVNHIFTIYLVLLLVLGLLLNGLALWVFCYRMHQWTETRVYMTNLAVADVCLLCSLPFVLYSLKYSTSDTPICQLSQGIYLVNRYMSISLVTAIAVDRYVAVRHPLRARELRSPRQAGAVCVALWVIVVTSLVLRWRLGIQEGGFCFSSQNRYNFSTTAFSLLGFYLPLAIVVFCSLQVVTALARRPATDVEQVEATQKATRMVWANLAVFIICFLPLHLILTVQVSLNLHTCAARNIFSRALTITAKLSDINCCLDAICYYYMAKEFQDASLRATASSTPHKSQDTQSLSLT

SEQ ID NO: 14 - protein, 307 aa, Mus musculus
MNSTTCNSTLTWPASVNNFFIIYSALLLVLGLLLNSVALWVFCYRMHQWTETRIYMTNLAVADLCLLCSLPFVLYSLKYSSSDTPVCQLSQGIYLANRYMSISLVTAIAVDRYVAVRHPLRARELRSPRQAAAVCVALWVIVVTSLVVRWRLGMQEGGFCFSSQTRRNFSTTAFSLLGFYLPLAIVVFCSLQVVTVLSRRPAADVGQAEATQKATHMVWANLAVFVICFLPLHVVLTVQVSLNLNTCAARDTFSRALSITGKLSDTNCCLDAICYYYMAREFQEASKPATSSNTPHKSQDSQILSLT

SEQ ID NO: 15 - protein, 119 aa, Mus musculus
MKLLASPFLLLLPVMLMSMVFSSPNPGVARSHGDQHLAPRRWLLEGGQECECKDWFLQAPKRKATAVLGPPRKQCPCDHVKGREKKNRHQKHHRKSQRPSRACQQFLKRCHLASFALPL

SEQ ID NO: 16 - protein, 119 aa, Rattus norvegicus
MKLLASPFLLLLTGMFTATVSSSPNQEVARHHGDQHQAPRRWLWEGGQECDCKDWSLRVSKRKTTAVLEPPRKQCPCDHVKGSEKKNRRQKHHRKSQRPSRTCQQFLKRCQLASFTLPL

SEQ ID NO: 17 - protein, 118 aa, Bos taurus
MKVLISSLLLLLPLMLMSVVSSSSHTGVARGQRDQRQASGRWLREGGQECECQDWFLRAPRRTLMAAPRLTKPCPCDHFKGRMKKTRHQRHHRKSNKPSRACQQFLTRCLLESFALPL

SEQ ID NO: 18 - protein, 119 aa, Felis catus
MRILISSLLLLLPLMLMPMVSSSRNPGVARGHRDQRQAPRRWLQEGSQECECKDWFLRAPKRKLMTVPGLPKKQCPCDHFKGSVKKTRHQRHHRKPNKHSRACQQFLTRCQLESFALPL

SEQ ID NO: 19 - protein, 119 aa, Macaca mulatta
MKVLISSFLLLLPLMLMSMVSSSLNPGVARGHRDQGQASRKWLQEGGQECECKDWFLRAPRRKVMTVSGLPKKQCPCDHFKGNVKKTRHQKHHRKPNKHSRACQQFLKQCQLRSFVLPL

SEQ ID NO: 20 - protein, 119 aa, Homo sapiens
MKVLISSLLLLLPLMLMSMVSSSLNPGVARGHRDRGQASRRWLQEGGQECECKDWFLRAPRRKFMTVSGLPKKQCPCDHFKGNVKKTRHQRHHRKPNKHSRACQQFLKQCQLRSFALPL

SEQ ID NO: 21 - protein, 119 aa, Pan troglodytes
MKVLISSLLLLLPLMLMSMVSSSLKPGVARGHRDRGQASRRWLQEGGQECECKDWFLRAPRRKLMTVSGLPKKQCPCDHFKGNVKKTRHQRHHRKPNKHSRACQQFLKQCQLRSFALPL

(19) United States
(12) Patent Application Publication
(10) Pub. No.: US 2016/0368995 A1
(43) Pub. Date: Dec. 22, 2016

(54) IDENTIFICATION OF CXCR8, A NOVEL CHEMOKINE RECEPTOR

(71) Applicant: THE REGENTS OF THE UNIVERSITY OF CALIFORINIA, Oakland, CA (US)

(72) Inventors: Albert Zlotnik, San Diego, CA (US); Jose L. Maravillasmontero, Gustavo A. Madero (MX); Amanda M. Burkhardt, Long Beach, CA (US)

(73) Assignee: The Regents of the University of California, Oakland, CA (US)

(21) Appl. No.: 15/023,493

(22) PCT Filed: Sep. 30, 2014
(86) PCT No.: PCT/US14/58451

§ 371 Date: Mar. 21, 2016

Related U.S. Application Data
(60) Provisional application No. 61/884,576, filed on Sep. 30, 2013.

(51) Int. Cl. C07K 16/28; A61K 39/00; C07K 14/52; C07K 14/715; G01N 33/74; C07K 16/24

(52) U.S. Cl. CPC C07K 16/2866; G01N 33/74; C07K 16/24; C07K 14/522; C07K 14/7158; A61K 39/0011; G01N 2333/726; G01N 2500/02; C07K 2317/76; A61K 2039/505

(57) ABSTRACT

Method of treating a subject for a disorder that correlates to increased CXCR8 signaling. The method includes disrupting the activation of receptor CXCR8 by ligand CXCL17 in the subject. In the method, the disrupting can include administering to the subject a substance that interferes with CXCL17 binding to CXCR8. Methods of screening, ligands, agonists, antagonists and vaccines involving the CXCR8/CXCL17 axis are also provided.

### STATEMENT REGARDING FEDERALLY SPONSORED RESEARCH OR DEVELOPMENT

This invention was made with Government support under Grant No. R01-AI093548 from the National Institutes of Health. The Government may have certain rights in this invention.

### REFERENCE TO SEQUENCE LISTING

A Sequence Listing is submitted herewith as an ASCII text file named “1279588SEQLIST”, created on Sep. 30, 2014 and having a size of 51 kilobytes. The Sequence Listing is incorporated by reference herein in its entirety.

### BACKGROUND

1. Field of the Invention

The invention relates to chemokine CXCL17 and its receptor CXCR8/GPR35.

2. Related Art

The human chemokine superfamily includes some 48 ligands and 19 known receptors. The receptors for most ligands have been identified, but some remain “orphans” (1). Chemokine (C-X-C motif) ligand 17 (CXCL17) was the last chemokine ligand to be described (2). The inventors previously reported that CXCL17 is a mucosal-associated chemokine that is significantly up-regulated in bronchoalveolar lavage of patients with idiopathic pulmonary fibrosis (IPF) (3). Importantly, it is also one of the few “orphan” chemokine ligands (the other being CXCL14) for which a receptor has not yet been identified (1).

### SUMMARY

Chemokines are a family of chemotactic cytokines that direct the traffic of leukocytes and other cells in the body. Chemokines bind to G protein-coupled receptors (GPCRs) expressed on the surface of target cells to initiate intracellular signaling cascades and induce chemotaxis. Although the cognate receptors of most chemokines have been characterized (4), the receptor for CXCL17, the most recent chemokine ligand to be reported, is still undefined. As described herein, it is shown that GPR35 is the receptor for CXCL17. CXCL17 is known to chemoattract macrophages and dendritic cells (2). GPR35 is expressed by/on CXCL17-responsive human monocytes, dendritic cells (DCs) and in the THP-1 monocytoid cell line. Additionally, transfection of GPR35 into Ba/F3 cells rendered them responsive to CXCL17 as measured by calcium mobilization assays. CXCL17 is a chemokine expressed in mucosal tissues (3); GPR35 expression mirrors this mucosal expression pattern. GPR35 also exhibits several structural features of chemokine receptors including a DRY box and a TxP motif. It is concluded that GPR35 is a novel chemokine receptor, and therefore suggest it should be named chemokine (C-X-C motif) receptor 8 (CXCR8). GPR35 has been associated with human disease; GWAS studies have linked it with inflammatory bowel disease (IBD) (5). Taken together, these observations strongly suggest that this novel mucosal chemokine CXCL17/CXCR8 axis represents an important target for therapeutic intervention in pathophysiological or inflammatory processes of the respiratory or digestive systems. The pairing is demonstrated in human, but counterparts in different species will similarly pair. Different species counterparts may be paired, and may show normal cross reactivity or may have different affinity or signaling capability compared to natural pairing.

In one aspect, a method of treating a subject for a disorder that correlates to increased CXCR8 signaling is provided. The method includes disrupting the activation of receptor CXCR8 by ligand CXCL17 in the subject. In the method: a) the disrupting can include administering to the subject a substance that interferes with CXCL17 binding to CXCR8; b) the disorder can be a gastrointestinal, respiratory, metabolic, infectious, or oncologic disorder, which in particular embodiments, can be a lung, digestive or reproductive system inflammatory disease; c) examples of such inflammatory diseases include, but are not limited to, Crohn's disease (CD), primary sclerosing cholangitis, ulcerative colitis, celiac disease, or irritable bowel syndrome (IBS), an ulcer, ischemic colitis, radiation colitis, celiacs disease, bronchopulmonary dysplasia, idiopathic pulmonary fibrosis, hypersensitivity pneumonitis, non-specific interstitial pneumonia, chronic obstructive pulmonary disease, pneumonia, asthma, bronchitis, emphysema, subclinical interstitial lung disease (subclinical ILD), cystic fibrosis, sarcoidosis, endometriosis, leiomyomas, adenomyosis, bacterial vaginosis, or infections or inflammation of the urethra; d) or any combination of a)-c).

In another aspect, a method of screening for a substance that disrupts the association between receptor CXCR8 and ligand CXCL17 is provided. The method includes adding CXCL17 to a cell expressing CXCR8, and measuring a reduction in CXCR8 signaling in the cell in the presence of the substance. For example, CXCR8 transfectants of the Ba/F3 cell line described in the Examples can be used to screen for agonists and antagonists of the CXCR8/CXCL17 interaction.

In a further aspect, a method of screening for a substance that disrupts the association between receptor CXCR8 and ligand CXCL17 is provided. The method includes adding CXCL17 to CXCR8, and measuring a reduction in CXCL17 binding to CXCR8 in the presence of the substance.

In these or other methods described herein, the substance can be: a) an antibody, or a fragment thereof, that binds to CXCL17 or CXCR8; b) a polypeptide exhibiting a natural, or a variant, sequence of CXCL17; c) a non-peptide conjugation variant of CXCL17; d) a small molecule that binds to CXCL17 or CXCR8; e) an aptamer that binds to CXCL17 or CXCR8; or any combination of a)-e).

In further aspects, the following are provided:

a) A ligand of CXCR8 is provided wherein said ligand binds selectively to the CXCR8 receptor. The ligand can be one that signals through said receptor, such as an agonist; signals less than 85%, 90%, 95%, or more of human CXCL17, such as an antagonist; is an inverse agonist (one that inhibits basal activity of CXCR8); is an allosteric modulator (one that alters the signaling activity of CXCR8 but does not interfere with the binding of the ligand (CXCL17); has at least about 85%, 90%, 95%, or more sequence identity to human CXCL17, such as a mutein; comprises a segment of at least 17, 19, 23, 27, 31 or more amino acids exhibiting at least 94% identity to human CXCL17; and/or binds to a primate CXCR8 receptor. The ligand can be one that: is in a sterile composition; is formulated for systemic or local administration; is in a therapeutic composition; is in a single dose container; and/or has at least 90% sequence identity to human CXCL17. In some embodiments that include at least about 85%, 90%, 95% or more sequence identity to human CXCL17, the embodiments do not include sequences identical to naturally occurring sequences of human CXCL17.

b) An antibody which binds selectively to a ligand of CXCR8 is provided. The antibody can block binding to the CXCR8 receptor; and/or block signaling by the CXCR8 receptor.

c) A receptor or binding protein for human CXCL17 is provided. The receptor or binding protein can: further signal upon binding of said human CXCL17; signal at least about 80% of signal upon binding of CXCL17 compared to human CXCR8; have at least about 95% identity to human CXCR8; and/or bind to primate CXCL17. In some embodiments that include at least about 95% or more sequence identity to human CXCR8, the embodiments do not include sequences identical to naturally occurring sequences of human CXCR8.

d) A method of inhibiting CXCL17 signaling through CXCR8 is also provided. The method includes contacting: a) CXCR8 (receptor) with a CXCL17 (ligand) antagonist; b) CXCL17 (ligand) with a blocking agent; and/or c) a cell expressing CXCR8 with a blocker of cell signaling. The CXCL17 (ligand) antagonist can be selected from: a) an antibody (or fragment thereof) which binds to CXCR8 (receptor) or species variant; b) a CXCL17 (chemokine) variant (e.g., which binds, but does not signal; including species variants and counterparts); or c) a small molecule compound. The blocking agent can be selected from: a) an antibody (or fragment thereof) which binds to CXCL17 (e.g., chemokine and blocks binding; including species variants); b) a fragment of the receptor, which can be a soluble portion of the receptor; and/or c) a small molecule compound. The blocker of cell signaling can be: a) RNAi, CRISPR, TALEN compound, e.g., of signaling pathway members; b) an antibody which blocks signaling pathway; or c) small molecule compound.

e) A method of inducing CXCR8 (receptor) signaling, said method comprising contacting said receptor with its cognate ligand, which can be CXCL17 or an agonist thereof. The agonist can be a polypeptide sequence variant of CXCL17 or a non-peptide conjugation variant of CXCL17 or fragments thereof.

f) A method of screening for said CXCL17 antagonists, wherein said screening uses a cell based assay using a fluorescent imaging plate reader (FLIPR) or related detection system including an assay selected from; FLIPR, cell based, biochemical, or other. In the method, said screening can be of one or more compounds which include: i) antibodies binding to CXCL17, including species variants or counterparts; ii) polypeptide sequence variants of CXCL17, including species variants; iii) non-peptide conjugation variants of CXCL17, e.g., glycosylation or other modifications; iv) small molecule antagonist candidates; or v) aptamer libraries.

g) A method of screening for said blocking agent described in (f), wherein said screening uses an assay such as FLIPR (on the World Wide Web at moleculardevices.com/Products/Instruments/FLIPR-Systems.html), cell based, or biochemical; i) antibodies binding to CXCR8 or species variants; ii) polypeptide sequence variants of CXCL17, e.g., soluble receptor fragments or species variants; iii) non-peptide conjugation variants of CXCL17, such as glycosylation or other modifications; iv) small molecule antagonist candidates; and/or v) aptamer libraries.

h) A method to screen for said CXCL17 antagonists or blocking agents, wherein CXCR8 transfectants of the Ba/F3 cell line are used to screen for agonists and antagonists of the CXCR8/CXCL17 interaction. In certain embodiments, said screening uses a cell based assay using a FLIPR or related detection system, which may be a cell based, biochemical, or other. In further embodiments, said screening is of one or more compounds which include: a) antibodies binding to CXCL17 or species variants; b) polypeptide sequence variants of CXCL17 or species variants; c) non-peptide conjugation variants of CXCL17, including glycosylation or other modifications; d) small molecule antagonist candidates; or e) aptamer libraries. A method is similarly provided wherein said screening uses a FLIPR, cell based, or biochemical assay. Additional embodiments include where said screening is of one or more compounds which include: a) antibodies binding to CXCR8 or species counterparts or variants; b) polypeptide sequence variants of CXCL17, including soluble receptor fragments and species counterparts or variants; c) non-peptide conjugation variants of CXCL17 including glycosylation or other modifications; d) small molecule antagonist candidates; or an aptamer library. In one particular embodiment, CXCR8 transfectants of the Ba/F3 cell line are used to screen for agonists or antagonists of the CXCR8/CXCL17 interaction.

i) Various gastrointestinal disorders that correlate to increased CXCR8 signaling and that can be treated by the methods include: a) Crohn's disease (CD), ulcerative colitis (UC), celiac disease, or irritable bowel syndrome (IBS), ischemic colitis, radiation colitis, celiac disease; b) stomach cancer, pancreatic cancer, colorectal cancer, or hepatocellular carcinoma, esophageal cancer, liver cancer, gallbladder cancer, biliary cancer, gastrointestinal stromal tumors; c) autoimmune hepatitis, primary biliary cirrhosis, other (non autoimmune) cirrhosis, primary sclerosing cholangitis, or liver fibrosis; or d) hepatitis C virus (HCV) mediated cirrhosis, peptic ulcers caused by Helicobacter pylori. See, e.g., Hauser, S. C. Mayo Clinic Gastroenterology and Hepatology Board Review, Fourth Ed. Mayo Clinic Scientific Press, 2013; Hawkey et al., Clinical and Gastroenterology and Hepatology, Second Ed. Wiley-Blackwell, 2012; and Yamada T. et al. Yamada's Handbook of Gastroenterology, 3rd Ed. Wiley-Blackwell, 2013.

j) Metabolic disorders that correlate to increased CXCR8 signaling and that can be treated by the methods include diabetes type 1, or diabetes type 2. See, e.g., Fonseca, V. A. Clinical Diabetes. Elsevier, 2012.

k) An oncologic metabolic disorder that correlates to increased CXCR8 signaling and that can be treated by the methods include leukemia, lymphoma, or glioblastoma or related brain tumor. See, e.g., Mughal, T. I. Understanding Leukemias, Lymphomas and Myelomas, 2^{nd} Ed. Informa 2012; and Kaye, A. H. and Laws E. R. Jr. Brain Tumors, 3^{rd} Ed. Elsevier 2012.

l) A respiratory disorder that correlates to increased CXCR8 signaling and that can be treated by the methods can be selected from: a) lung cancer (6), including small (7) or non-small cell lung cancer (8) or mesothelioma (9) (malignant); b) idiopathic pulmonary fibrosis (10), hypersensitivity pneumonitis (11), or non-specific interstitial pneumonia; c) a respiratory disease associated with interstitial lung disorders including autoimmune diseases like rheumatoid arthritis or scleroderma; d) chronic obstructive pulmonary disease (COPD) (12), bronchopulmonary dysplasia (BPD) (13), or asthma (14); and/or e) other respiratory cancers, including trachea cancer, cancer of the larynx, cancer of the esophagus, cancer of the bronchus, or nasal/sinus cancer. See, e.g., Judd, S, J, Respiratory Disorders Sourcebook, 2^{nd} Ed. Health Reference Series, 2012; and Lechner, A. Respiratory, An integrated approach to disease; McGrawHill LANGE, 2012.

m) The administering can be a) topical, local, or systemic; b) inhaled as an aerosol or mist; or c) in combination with another therapeutic.

n) A vaccine comprising a CXCL17 agonist, e.g., as an adjuvant and/or agonist, is provided, or comprising a positive allosteric modulator, that is, a molecule without agonist or antagonist activity (for CXCL17) that alters the signaling ability of the receptor (CXCR8) is provided. The vaccine can include protective antigens such as those in vaccines for hepatitis B, human papilloma virus, DPT, and/or measles virus. In some cases, a target antigen is a tumor associated antigen (including tumors from the following cancers: lung, pancreatic, colorectal, prostate, breast, hepatocellular carcinoma, soft tissue sarcoma, and/or glioblastoma), or in disperse leukemias and lymphomas. The vaccine can be used for a cancer selected from lung, pancreatic, colorectal, prostate, breast, hepatocellular carcinoma, soft tissue sarcoma, or glioblastoma. The vaccine can be administered to a subject. See, e.g., Plotnik, S. A. et al. Vaccines, 6^{th} Ed. Elsevier 2012. In other embodiments, the vaccine may include an antagonist of CXCL17, at the right concentration, capable of inhibiting the recruitment of tolerogenic cells.

o) A method of mediating elevated blood pressure in a subject, said method comprising administering a suitable amount of a CXCR8 agonist to mediate said blood pressure. The elevated blood pressure can be hypertension in some embodiments. The agonist can be selected from: a) recombinant human CXCL17; b) a polypeptide variant of human CXCL17 (including species variants); c) non-peptide conjugation variants of CXCL17 (e.g., glycosylation or other modifications).

p) A method of recruiting macrophages or dendritic cells, said method comprising administering a CXCR8 antagonist (e.g., and harvesting said cells); which may further comprise administering a CCR2 agonist, like CCL2, defined as such a molecule that elicits a calcium flux in a cell expressing CCR2.

q) A method that uses CXCR8 as a marker of cells involved in the pathogenesis of human diseases including gastrointestinal, metabolic and respiratory diseases and cancer, a biomarker of metastatic cells of leukemias, lymphomas, stomach cancer, colorectal cancer or pancreatic cancer, a biomarker of metastatic cells of lung cancer including small or non-small cell lung cancer or malignant mesothelioma, a biomarker of subclinical interstitial lung disease (subclinical ILD), or prognostic biomarker of cells that infiltrate gastrointestinal or respiratory system cancers.

r) A method of treating or preventing atherosclerosis (see, e.g., George, S. J. Atherosclerosis: Molecular and Cellular Mechanisms, Wiley-Blackwell 2012), or treating or preventing multiple sclerosis (see, e.g., Holland, N. et al. Multiple Sclerosis, 4^{th} Ed. Demos Health, 2012), said method comprising administering to a subject an effective amount of: a) a CXCR8 antagonist or; inhibitor of CXCR8 expression; or b) a CXCL17 antagonist or inhibitor of CXCL17 expression. The CXCR8 antagonist can be selected from: a) an antibody binding to CXCR8 (or species variants; e.g., binds but sends no signal); b) polypeptide sequence variants of CXCL17 (e.g., soluble receptor fragments; species variants); c) non-peptide conjugation variants of CXCL17 (e.g., glycosylation or other modifications); d) small molecule antagonist; or e) an aptamer. The inhibitor of CXCR8 expression or downstream signaling can use an RNAi, CRISPR, TALEN compound or the like. The CXCL17 antagonist can be selected from: a) an antibody binding to CXCL17 (or species variants; binds but sends no signal); b) a polypeptide sequence variant of CXCL17 (including species variants); c) a non-peptide conjugation variant of CXCL17 (e.g., glycosylation or other modifications); d) a small molecule antagonist; or e) an aptamer. The inhibitor of CXCL17 expression or signaling can use an RNAi, CRISPR, TALEN compound or the like.

s) A method of inhibiting CXCL17 signaling through CXCR8, said method comprising reducing CXCR8 receptor using an RNAi, CRISPR, or TALEN compound which inhibits expression of CXCR8 (receptor). Also, a method of inhibiting CXCL17 signaling through CXCR8, said method comprising reducing CXCL17 using an RNAi, CRISPR, or TALEN compound which inhibits expression of CXCL17 [ligand].

t) A method of isolating CXCR8-expressing cells, comprising mixing an anti-CXCR8 antibody with a peripheral blood mononuclear cell preparation, and separating CXCR8 positive cells bound by the antibody. In the method, the anti-CXCR8 antibody can be a monoclonal antibody, neutralizing antibody, or humanized antibody, or combination thereof; the separating can be by fluorescence-activated cell sorting; and/or the separating can be by magnetic bead isolation.

In some embodiments of the methods, including the method of treating a subject for a disorder that correlates to increased CXCR8 signaling, the substance, agonist or antagonist does not include the following: kynurenic acid, 2-Acyl lysophosphatidic acid, cromolyn, dicumarol, luteolin, niflumic acid, NPPB, pamoates and pamoic acid, quercetin, thyrphostin-51, zaprinast, ML144, ML145, or CID-2765487.

The molecule GPR35 is also referred to as CXCR8 throughout this application.

The subject can be a human or other animal, and will typically be a primate or mammal.

Sequences of CXCL17 from various species have the following accession numbers (all incorporated by reference herein): HGNC: 19232 (Human CXCL17) (HUGO Gene Nomenclature Committee database; Homologs: MGI:2387642 (mouse Cxcl17) (MGI database); RGD:1304717 (Rat Cxcl17) (RGD database); nucleotide sequence: RefSeq: NM198477 (NCBI Reference Sequence Database); protein sequence: UniProtKB:Q6UXB2 (UniProt Knowledgebase). See also GENBANK, NCBI, dbest, Swiss-prot, Unigene, Refseq, nr-aa, PRF, or PDBSTR.

Sequences of CXCR8/GPR35 from various species have the following accession numbers (all incorporated by reference herein): HGNC: 4492 (Human GPR35) (HUGO Gene Nomenclature Committee database; Homologs: MGI: 1929509 (mouse Gpr35) (MGI database); RGD: 1309404 (Rat Gpr35) (RGD database); nucleotide sequence: RefSeq: NM 001195382 (NCBI Reference Sequence Database); protein sequence: UniProtKB:Q9HC97 (UniProt Knowledgebase). See also GENBANK, NCBI, dbest, Swiss-prot, Unigene, Refseq, nr-aa, PRF, or PDBSTR.

### BRIEF DESCRIPTION OF THE DRAWINGS

For a more complete understanding of the present invention, reference is now made to the following descriptions taken in conjunction with the accompanying drawings, in which:

FIG. 1 is a panel of results showing that THP-1 cells are responsive to CXCL17. FIG. 1A, THP-1 cells were tested in CXCL17-directed chemotaxis transwell assays, both under resting or PGE2 pre-treated conditions; additionally, these cells were also tested in the same way after a pre-treatment with Bordetella pertussis toxin (PTX). The bars show the total number of recovered cells (chemotaxed) in the lower chamber of the transwell plate. FIG. 1B, representative calcium flux response of THP-1 cells (loaded with the Ca⁺² sensitive dyes), whether resting or PGE2 treated, when stimulated with 100 nM of CXCL17. n=2. C, for desensitization of the CXCL17 receptor expressed by the THP-1 cells, 100 nM of CXCL17 or CCL2 were alternative added at the indicated points to induce cellular calcium flux responses. Representative graphs showed, n=3.

FIG. 2 is a panel of schematic drawings representing typical chemokine receptor features. FIG. 2A, localization of the GPR35 gene in the distal region of the long arm of the human chromosome 2; as depicted, it is possible to see the neighboring CXCR7 gene in the proximity. FIG. 2B, phylogenetic analysis of the protein sequences of the known chemokine receptors showing that the most closely related member to GPR35 is CXCR7. FIG. 2C, alignment of protein sequences of the most abundant chemokine receptors in resting monocytes accordingly to the BIGE (CCR1 (SEQ ID NO. 3), CCR2 (SEQ ID NO. 1), CCR5 (SEQ ID NO. 2) and CXCR4 (SEQ ID NO. 4)) plus CXCR7 (SEQ ID NO. 5) and GPR35 (SEQ ID NO. 6). Conservation levels are showed as darker gray shades in the background of each amino acid. The seven transmembrane (TM) domains are showed. The boxes indicate the DRY box and the TxP motif; the arrow head depicts the conserved aspartic acid at the second TM region. A consensus sequence (SEQ ID NO. 7) is also shown.

FIG. 3 is a panel of results showing that GPR35 is expressed in THP-1 cells. FIG. 3A, relative expression of GPR35 in resting or PGE2 treated THP-1 cells measured by qRT-PCR. The data were normalized with the relative expression of GAPDH in the samples. Representative experiment, n=2. FIG. 3B, expression of GPR35 protein measured by flow cytometry comparing the expression of GPR35 in resting THP-1 cells (which are positive) and Ba/F3 cells (which are negative) versus the isotype control (rabbit IgG).

FIG. 4 is a panel of results showing that CXCL17 induces cellular calcium mobilization through GPR35. FIG. 4A, calcium flux responses in mock or GPR35 transiently transfected Ba/F3 cells loaded with Ca⁺² sensitive dyes, upon the addition of CXCL17 [100 nM]. Representative graph of 3 experiments performed. FIG. 4B, dose-response relationship observed in the GPR35 transfected Ba/F3 cells upon the addition of different amounts of CXCL17.

FIG. 5 is a table (Table 1) showing the relative expression of GPR35 in different cells or tissues of the human body from the BIGE database. The data represent microarray analyses and the average intensity refers to the ability of the probeset corresponding to GPR35 to hybridize to mRNA corresponding to each of these tissues/cells.

FIG. 6 is a graph showing that expression of GPR35 in HEK293 cells make them responsive to CXCL17. HEK293 cells were transfected with the expression vector containing the human GPR35 coding sequence and were analyzed 72 h post-transfection with the Ca⁺² mobilization approach described in material and methods section. The cells were stimulated with 100 ng of CXCL17 added at the marked time point.

FIG. 7 is a graph showing that the mucosal chemokines CXCL14 or CCL28 do not induce GPR35 signaling. GPR35/CXCR8 Ba/F3 transfected cells were tested for Ca⁺² mobilization with human CXCL17, CXCL14 and CCL28 (at a concentration of 100 nM), independently added at the indicated time point.

FIG. 8 is a table (Table 2) showing GPCRs expressed by human monocytes. FIGS. 8A and 8B each include a part of the table.

FIG. 9 is a table (Table 3) showing results of radioligand displacement studies of several chemokine receptors (n.d. means not detectable).

FIG. 10 is a table (Table 4) showing results of chemokine-induced β-arrestin recruitment.

FIG. 11 is a panel of graphs showing expression of CXCR8 and CXCL17 in Salmonella infected mice.

FIG. 12 is a graph showing that CXCR8 is elevated in a mouse model of ulcerative colitis.

FIG. 13 is a graph showing that CXCR8:CXCL17 mediated chemotaxis is comparable to CCR2, a key macrophage chemoattractant.

FIG. 14 is a sequence alignment of CXCR8 from various animals. The alignment is performed using CLUSTAL Omega multiple sequence alignment tool (Sievers and Higgins, Clustal Omega accurate alignment of very large numbers of sequences. Methods Mol Biol. 2014; 1079:105-16). In the figure, consensus residues are shown, where (*) indicates complete sequence similarity at a particular residue while (.) and (:) indicate partial sequence similarity at a particular residue. No symbol indicates no significant sequence similarity at that particular residue. The CXCR8 sequence from Felis catus (SEQ ID NO. 8), Bos taurus (SEQ ID NO. 9), Homo sapiens (SEQ ID NO. 10), Pan troglodytes (SEQ ID NO. 11), Macaca mulatta (SEQ ID NO. 12), Rattus norvegicus (SEQ ID NO. 13) and Mus musculus (SEQ ID NO. 14) are shown. FIGS. 14A and 14B each include a part of the alignment.

FIG. 15 is a sequence alignment of CXCL17 from various animals. The alignment is performed using the CLUSTAL Omega multiple sequence alignment tool. In the figure, consensus residues are shown, where (*) indicates complete sequence similarity at a particular residue while (.) and (:) indicate partial sequence similarity at a particular residue. No symbol indicates no significant sequence similarity at that particular residue. The CXCL17 sequence from Mus musculus (SEQ ID NO. 15), Rattus norvegicus (SEQ ID NO. 16), Bos taurus (SEQ ID NO. 17), Felis catus (SEQ ID NO. 18), Macaca mulatta (SEQ ID NO. 19), Homo sapiens (SEQ ID NO. 20) and Pan troglodytes (SEQ ID NO. 21) are shown.

FIG. 16 is a graph showing chemotactic responses to CXCL17 following mild crosslinking of membrane proteins.

### DETAILED DESCRIPTION

The following application is incorporated by reference herein: U.S. Provisional Patent Application No. 61/884,576, filed on Sep. 30, 2013.

Chemokines and chemokine receptors are known for controlling the migration of cells within the body but can also alter the homeostasis of the responding cells that express the appropriate receptor for a given ligand (1, 15). Embodiments of the present invention are based, in part, on the identification of the cognate receptor for the chemokine CXCL17 which is represented by the G-protein-coupled receptor GPR35. As a consequence of this identification GPR35 can now be renamed CXCR8 as per the established guidelines of chemokine receptor nomenclature (1).

### CXCL17 Chemokine (Including Species Counterparts)

The chemokine CXCL17 exists in human (Locus tag UNQ473/PRO842) (Q6UXB2(UniParc)) (NP_940879.1), Mouse (NCBI gene ID: 284340) (NP_705804.2), Chimpanzee (XP_001154726.1), and other mammals including the dog, elephant and gorilla. CXCL17 is likely to exist in many species and can be identified by BLAST searches of comprehensive databases like Swiss-Prot or NR-AA (see for example: on the World Wide Web at genome.jp/tools/blast/). Natural sequences may in many cases be substituted by variants thereof, including in certain embodiments at least about 80% identity, about 85%, or about 90% identity or more, including at least about 95% or 100% identity. For example a segment of comparison may be about 95% of the amino acids in length, or about 90%, 85%, or 80% of the amino acids of the length for comparison. The length of comparison may be at least about 20, 30, 40, 50, 55, 60, 65, 70, or 75 amino acids. The variants may conserve particular physicochemical or functional features as the prevailing natural sequence, while other variants may have modified combinations of structural and functional features. In some embodiments, the variants do not include sequences identical to naturally occurring human CXCL17 or CXCR8 sequences, or naturally occurring CXCL17 or CXCR8 sequences of other animals. Truncated versions, or fusions with other segments are provided, which exhibit a function as described. Embodiments of the present invention allow for evaluating function corresponding to structural changes.

### CXCR8 Chemokine Receptor (Including Species Counterparts)

CXCR8 chemokine receptor (which include species counterparts) are described. Variants of the sequence with appropriate functions, are provided herein. In particular, variants will typically retain at least about 80%, 85%, 90%, and 95% or more identity in sequence to the natural sequences. In other embodiments, variants will have regions of differing identity, and may include segments of various lengths, e.g., about 20, 30, 40, 50, 70, 100 or more amino acids of specific identity, e.g., 100%, about 95%, 90%, 85%, 80% or lesser identity to the reference sequence. Preferred human sequences are described above, and include accession numbers: NP_001182310; Q9HC97; BC095500.

### CXCL17 Chemokine and CXCR8 Chemokine Receptor Pairing (Ligand-Receptor Pairing)

Embodiments of the invention describe the identification of the receptor for the CXCL17 chemokine. It is a G-protein coupled receptor GPR35, which can now be renamed CXCR8. The importance of this discovery is that both are proteins expressed in mucosal sites where they recruit various cells of the immune system, including macrophages, monocytes and dendritic cells to maintain homeostasis and to regulate inflammatory responses in these tissues, among other functions. There are a number of inflammatory conditions in these tissues that cause human disease, and regulating the CXCL17/CXCR8 axis is therefore important to get therapeutic benefit.

### Pairing Function (Ligand Production, Receptor Binding, Signaling, Effector Functions)

Given that there is a large number of class A GPCRs in the human genome (more than 273) it is not easy to identify a novel chemokine receptor. There are few chemokines that do not yet have receptors identified (the other one is CXCL14) (1). The inventors consider that the identification of CXCR8 was difficult and non obvious because it first involved the identification of all the GPCRs expressed by responding cells, and then testing each one until the correct receptor through which CXCL17 signals was found. The identification of CXCR8 allows us to predict that it will signal and mediate effector functions of CXCL17. Both CXCL17 and CXCR8 are over-expressed under inflammatory conditions, and this is a common feature of other chemokine/receptor axes that participate in inflammatory responses (16). Following the initial calcium flux that is triggered by the initial binding of the chemokine to its receptor, there are a number of phosphorylation steps that lead to changes in the cytoskeleton of the cell and prepares it for migratory responses (16).

### Ligand Analog Structures, Agonists and Antagonists

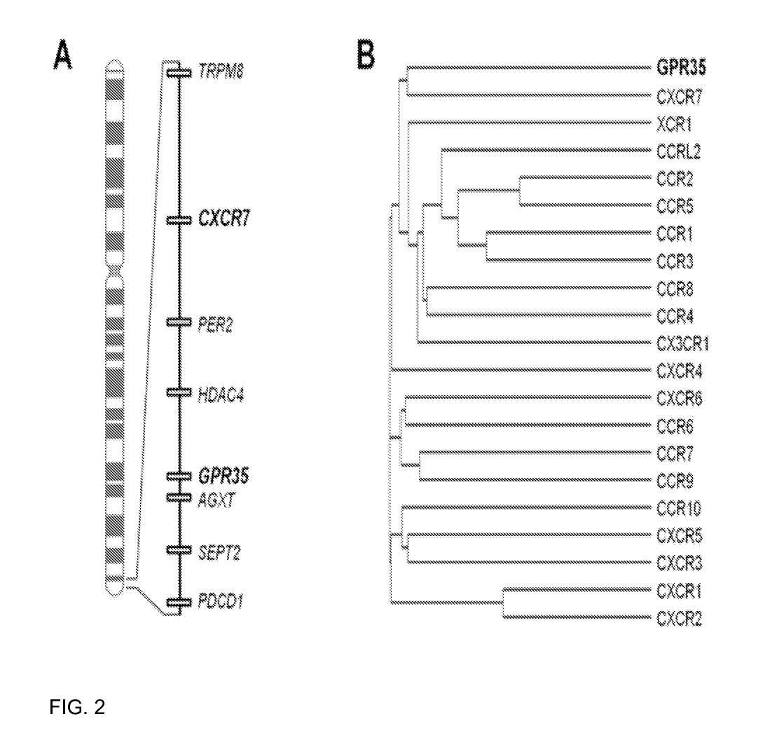

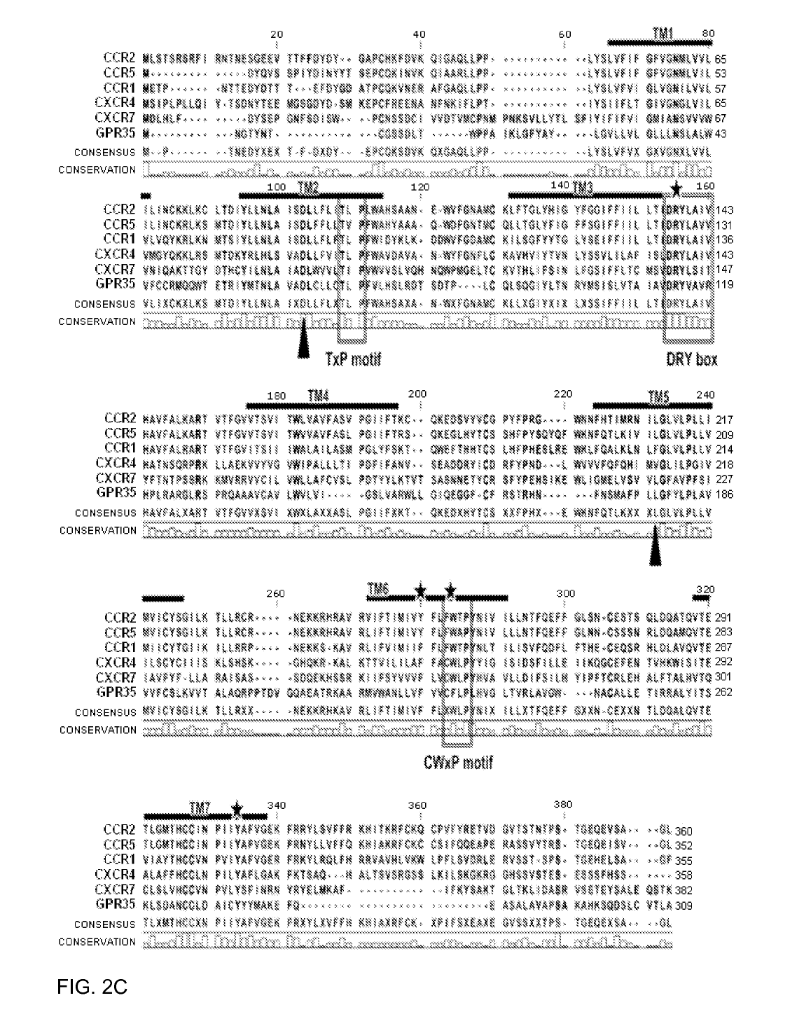

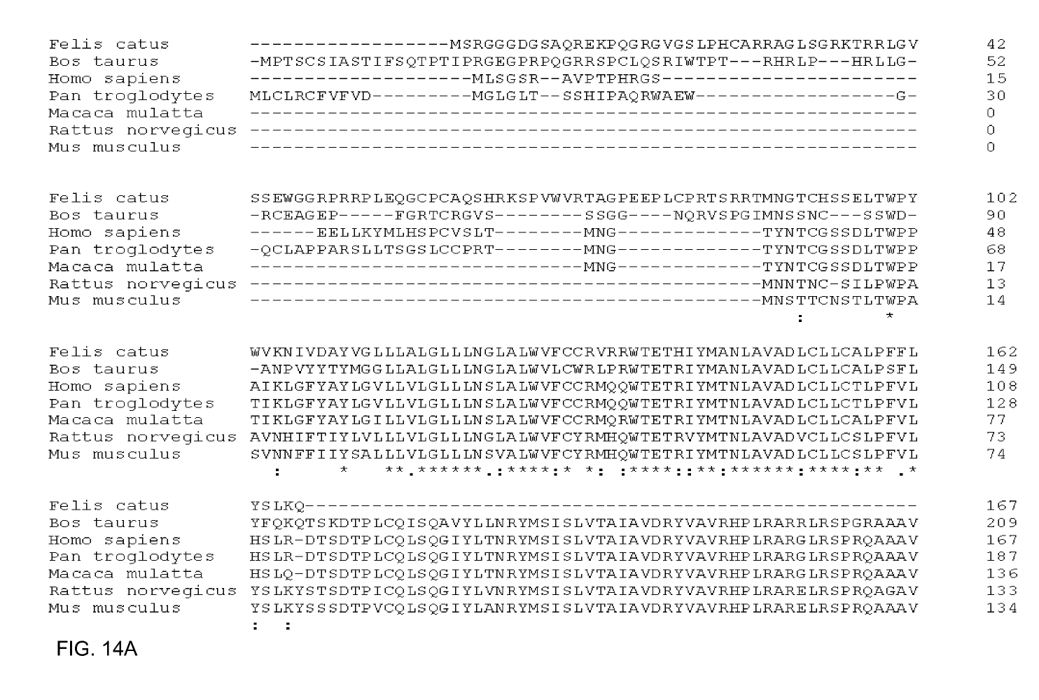

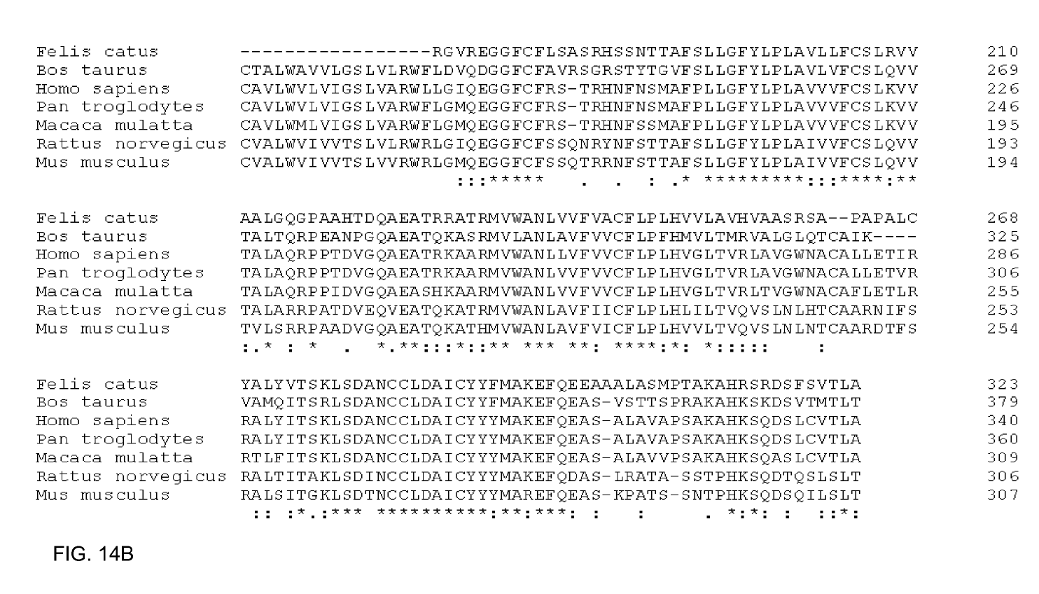

It can be predicted that the binding of CXCL17 to CXCR8 can be eliminated by introducing mutations in the protein interacting segments, or binding sites of these proteins. The ligand binding site of CXCR8 should include the NH₂ terminus about 1-25 and exposed sites of the GPCR loops that face the exterior of the cell which may include residues about 73-about 105, and about 150 to about 175 of the sequence of accession number NP_005292. FIG. 2C shows the sequence homology between CXCR8 (GPR35) and several other human chemokine receptor molecules. Consensus sequence is shown and the relative extent of conservation between all the receptors. Domains common to the GPCR family such as the seven transmembrane domains (TM), the TxP motif and the DRY box are indicated. FIG. 14 is a sequence alignment of CXCR8 from various animals.

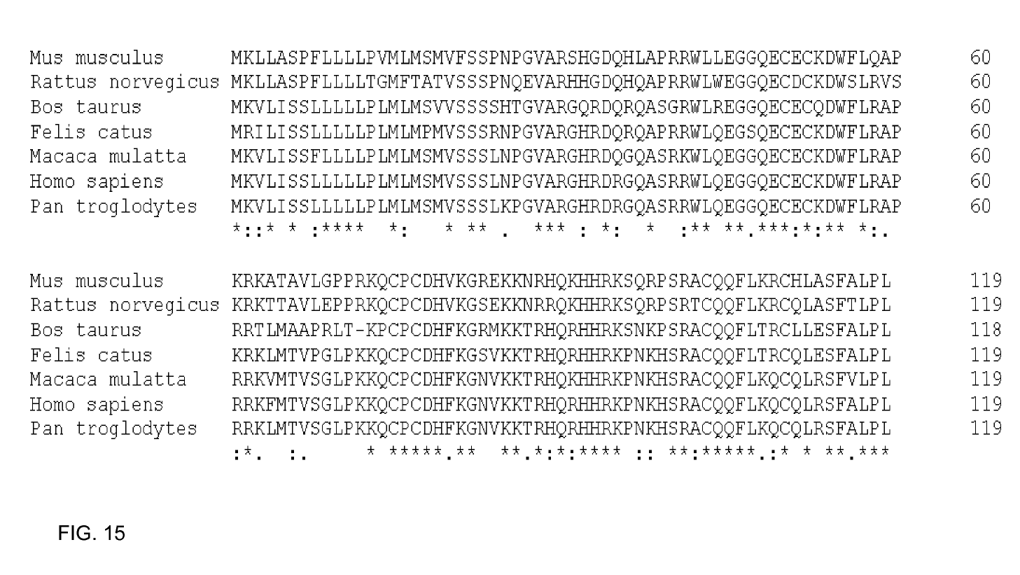

Similarly, CXCL17 mutants can be constructed by mutations in the core of the chemokine, those areas between the 2 disulphide bridges characteristic of chemokines CXCL17 exhibits some original structure, which partly explains why it was the most recent chemokine discovered (2), so it is possible that mutations in other areas, for example, residues about 23 to about 49 and about 104 to about 119 of the sequence of accession number NP_940879 could render it incapable of binding CXCR8. FIG. 15 is a sequence alignment of CXCL17 from various animals. Nevertheless mutagenesis methods and analysis are common techniques familiar to those skilled in the art so there should be no problem identifying empirically how function is affected by structural variations in the CXCL17 and CCR8 proteins.

The CXCL17 mutant because of its soluble nature will be more useful to use as an antagonist (if it binds but does not signal) or alternatively, some mutants may show enhanced binding and signaling and may have other uses in the recruitment of specific responding cells.

Antibody structures, against ligand, against receptor; fragments, aptamer; non-polypeptide structures (e.g., non-peptide linkages; modified polypeptides); RNAi, CRISPR, TALEN compounds affecting receptor/ligand interactions; screening for receptor binding (use ligand as positive control), and compound libraries, are embodiments of the invention.

Antagonists against CXCR8 or CXCL17 include certain antibodies against these proteins, as well as mutant CXCL17 protein. It is also possible to use small molecule antagonists that can be identified by using BA/F3 cells transfected with CXCR8 for use in calcium-flux based screening assays like those based on FLIPR technology (17). The FLIPR (fluorescent imaging plate reader) assay uses trans-laser illumination of multiwell cell culture plates, and light emissions are detected from above. Typically, cells are loaded with a Ca²⁺ indicator fluorophore (such as Fluo3) and the emitted fluorescence indicates relative Ca²⁺ levels within the illuminated cells. Test compounds can be added from multiwell plates containing premeasured compounds directly to the assay plates containing cells. This configuration enables continuous measurement of cell Ca²⁺ levels before and after addition of test compounds, and allows for measurement of compound activities toward the signaling capacity of the test cells. Various compound libraries can be screened using these methods including those used by companies like Merck, Lilly, Pfizer, etc. See for examples (on the World Wide Web at enzolifesciences.com/welcome/compound-libraries/).

Of particular importance, the pairing provided here serves as a positive control for a screening assay. It can be used quantitatively, e.g., to evaluate the specific activity and pharmacological signaling of natural interaction. Specific activity of variant forms can be evaluated as partial agonists or partial antagonists. Different forms may have differing spectra of activity across different receptor variants found in various therapeutic subpopulations. Thus, different variants may have greater or lesser variation in responsiveness to heterogeneous target populations, e.g., expressing different receptor isotypes.

Other features that can affect binding or other pharmacology include glycosylation, methylation, acetylation, or other modifications. In certain embodiments, a non-peptide linkage of peptide sequences may achieve the same function to link two peptides. These include aptamers which are oligonucleic acid or peptide molecules that bind to a specific target molecule. Other possible inhibitors of the CXCL17/CXCR8 interaction include RNAi (interference RNA used to inhibit gene expression) (see, e.g., Cheng, K., and Mahato R. I. Advanced delivery and therapeutic applications of RNAi, Wiley, 2012). RNAi molecules introduced into cells will lead to the destruction of cellular RNA through a normal cell pathway and thereby prevent the expression of the protein encoded by a DNA sequence and the resultant mRNA. RNAi molecules are frequently used to reduce or eliminate the expression of targeted molecules in biological research. In a therapeutic setting, mRNA could be used to reduce or eliminate the expression of CXCR8 or CXCL17 proteins, thereby reducing the signaling and biological effects of CXCL17 and CXCR8. CRISPR, TALEN compounds, and the like affecting receptor ligand interactions may also be used (see on the World Wide Web at sciencemag.org/content/341/6148/833.full). CRISPR and TALEN molecular technologies use DNA-binding proteins (TALEN) or RNA molecules (CRISPR) to guide associated nuclease molecules to a specific DNA sequence in the genome. The nuclease introduces double stranded DNA breaks. In the presence of introduced locus-specific homology arms, mutations, deletions and insertions can be introduced at the target site. Such techniques could be used in a research or clinical setting to decrease or increase the signals normally driven by the interaction of CXCR17 and CXCR8.

### Diagnostic Uses of Pairing; Label One, Assay for Other, Functional Sensitivity, Etc.

Selective interaction will allow for using one of the pair to be used to detect the partner. Label of one will allow for identifying the partner. The label may include radioactive, isotope, fluorescent, or other. Antibodies may also be used to detect and evaluate body, organ, and tissue distribution. These distribution patterns may be useful as diagnostic evaluations, e.g., for the clinical indications described.

### Diagnostic Methods (e.g., Chemokine/Receptor Based Patient Subsetting)

CXCL17 and CXCR8 may also be useful as biomarkers for specific diagnostic uses. These include the ability to quantify CXCR8+ cells or subtypes in the blood of patients, the numbers or types of which may be altered in various pathological conditions, or the concentration of CXCL17 in bodily fluids that can be measured by ELISA or similar methods. See e.g., Pagana and Pagana, Mosby's Manual of Diagnostic and Laboratory Tests Fourth Ed. Mosby Elsevier 2013. CXCL17 and/or CXCR8 may also be used as biomarkers of subclinical interstitial lung disease (subclinical ILD).

### Therapeutic Methods Using Chemokine or Receptor (Clinical Indications)

It is expected that agonists or antagonists of the CXCL17/CXCR8 interaction will be useful for various therapeutic indications based on the expression pattern of these proteins which includes the mucosal sites of the respiratory, gastrointestinal and female reproductive systems. These proteins will be involved in the pathogenesis of several cancers, including glioblastoma or other brain cancers, as well as multiple sclerosis and they will also likely be involved in the control of blood pressure.

The subject can be, e.g., a mammal, a primate, a human, a farm animal, a companion animal, a human, a poultry species, a cow, a horse, a goat, a cat, a sheep, a rodent, a dog, a pig, a chicken, a duck, a turkey, a quail, or a goose. A display or exhibition animal may also be treated, e.g., zoo or performing animal, including pinipeds, whales, dolphins, lions, tigers, and other veterinary subjects.

Combination Therapies (in Combination with Another Treatment)

A preferred use of an embodiment of the invention will be to control inflammation. Here, agonists or antagonists of the CXCL17/CXCR8 interaction may be used with other established anti-inflammatories including non-steroidal anti-inflammatories, aspirin, or anti-TNFα agents like Humira, Remicade, or Enbrel. In particular, combinations with therapeutic antibodies are provided. Other indications may be treated in classical methods, whose efficacy may be synergistic with the methods provided herein.

Making chemokine, analogs (recombinant, chemical linkages, glycosylation, etc.); making receptor analogs; nucleic acids encoding analogs, including expression constructs, plasmids; cells, animals comprising nucleic acids (eukaryotes, prokaryotes).

Standard methods for producing and making the ligands, receptors, and variants can be applied. Standard recombinant methods can be developed, including design of recombinant nucleic acids encoding constructs. See, e.g., Thompson D. A. Cell and Molecular Biology Manual 2011. Expression vectors, e.g., with promoters operably linked to coding regions, can be devised. Cells comprising the vectors are provided, including both prokaryote cells and eukaryote cells. Compatible expression methodologies can also be developed.

Typically, a polynucleotide that encodes the cell wall degrading polypeptides is placed under the control of a promoter that is functional in the desired host cell. An extremely wide variety of promoters is well known, and can be used in expression vectors of embodiments of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Accordingly, embodiments the invention provide expression cassettes into which the nucleic acids that encode the relevant functional polypeptides are incorporated for high level expression in a desired host cell (see, e.g., Ream W and Field K. G. Molecular Biology Techniques. Academic Press. 2012).

Substantially pure compositions of at least about 70, 75, 80, 85, 90% homogeneity are preferred, and 92, 95, 98 to 99% or more homogeneity are most preferred. The purified polypeptides may also be used, e.g., as immunogens for antibody production, which antibodies may be used in immunoselection purification methods.

### Formulations

Different formulations can be used (sterile, buffered, slow release, controlled release, stabilizers, ointments, etc.) depending on the optimal route of administration. See, e.g., Niazi S. K. Handbook of Pharmaceutical Manufacturing Formulations Informa Healthcare 2012. As with anti-inflammatories, agonists or antagonists of the CXCL17/CXCR8 interaction can be used in combination with other established drugs to optimize therapeutic outcomes. In addition, the compound(s) can be used in combination with other therapeutics in a single formulation strategy. Pharmacological variants can be used to obtain desired pharmacokinetic outcomes (secretion, half life, solubility or optimize excretion routes).

The exact dose will depend on the purpose of the treatment, and will be ascertainable by one skilled in the art using known techniques. See, e.g., Ansel, et al., Pharmaceutical Dosage Forms and Drug Delivery; Lieberman (1992) Pharmaceutical Dosage Forms (vols. 1-3), Dekker, ISBN 0824770846, 082476918X, 0824712692, 0824716981; Lloyd (1999) The Art, Science and Technology of Pharmaceutical Compounding; and Pickar (1999) Dosage Calculations. As is known in the art, adjustments for protein degradation, systemic versus localized delivery, and rate of new protease synthesis, as well as the age, body weight, general health, sex, diet, time of administration, drug interaction, and the severity of the condition may be necessary, and will be ascertainable with some experimentation by those skilled in the art.

Various pharmaceutically acceptable excipients are well known in the art. As used herein, “pharmaceutically acceptable excipient” includes a material which, when combined with an active ingredient of a composition, allows the ingredient to retain biological activity and without causing disruptive reactions with the subject's immune system. Such may include stabilizers, preservatives, salt or sugar complexes or crystals, and the like. See, e.g., Niazi S. K. Handbook of Pharmaceutical Manufacturing Formulations Informa Healthcare 2012.

Exemplary pharmaceutically carriers include sterile aqueous of non-aqueous solutions, suspensions, and emulsions. Examples include, but are not limited to, standard pharmaceutical excipients such as a phosphate buffered saline solution, water, emulsions such as oil/water emulsion, and various types of wetting agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. In other embodiments, the compositions will be incorporated into solid matrix, including slow release particles, glass beads, bandages, inserts on the eye, and topical forms. Administration routes may include the following: topical, systemic, respiratory, oral, eye, implant, vaginal, anal, suppository, devices with control release, sublingual, buccal, nasal, inhalation, parenteral, intraorgan, subcutaneous, intradermal, intramuscular, intravenous, and the like.

The present invention may be better understood by referring to the accompanying examples, which are intended for illustration purposes only and should not in any sense be construed as limiting the scope of the invention. Although the description of the invention has included description of one or more embodiments and certain variations and modifications, other variations and modifications are within the scope of the invention, e.g., as may be within the skill and knowledge of those in the art, after understanding the present disclosure. All publications, patents, patent applications, Genbank numbers, and websites cited herein are hereby incorporated by reference in their entireties for all purposes.

### Example 1

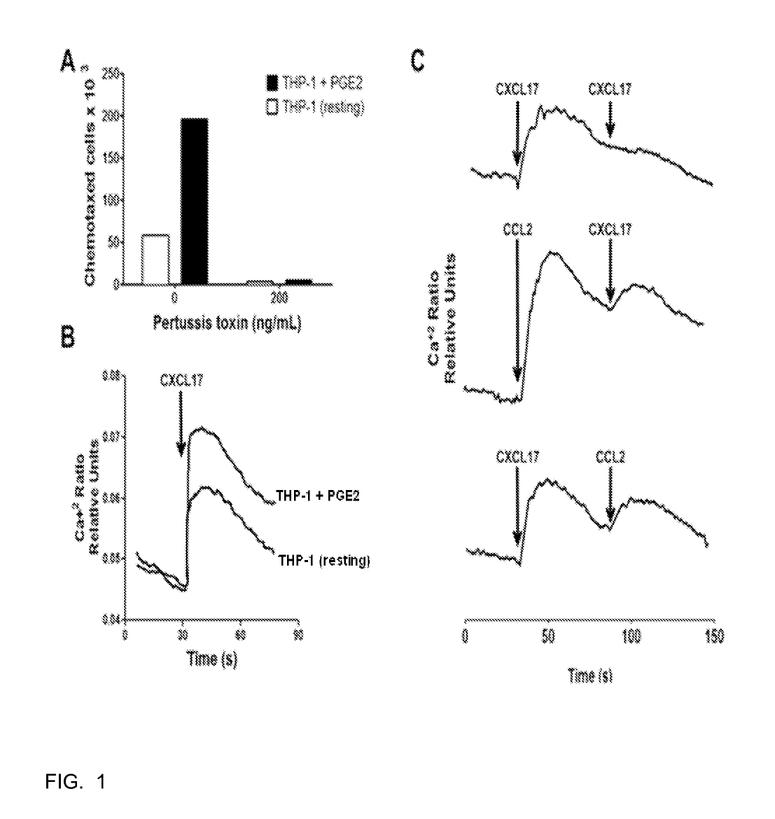

We began by identifying cells that responded to CXCL17. To this end, we measured the response of multiple cell lines to recombinant CXCL17 using transwell-based chemotaxis assays. One of the best chemotactic responses induced by CXCL17 was observed with the THP-1 human cell line (FIG. 1A). THP-1 cells were derived from a patient with acute monocytic leukemia (18) and have been widely used for monocyte/macrophage studies.

Given that CXCL17 is known to recruit monocytes and dendritic cells (2) we concluded that THP-1 cells must express the CXCL17 receptor. Importantly, we also found that the response of the THP-1 cells to CXCL17 increased following treatment with prostaglandin E2 (PGE2) (FIG. 1A). Previous reports have made similar observations in the chemotactic responses of THP-1 to other chemokines (for example, CXCL14), following PGE2 treatment (19). Furthermore, the chemotactic response of the THP-1 cells is sensitive to Bordetella pertussis toxin (PTX) (FIG. 1A). PTX is known to inhibit G_{αi/o} protein signaling pathways (20-21). Since most chemokine receptors elicit their response via G_{αi/o} proteins, this observation suggested that the CXCL17 receptor activates the same signaling pathways.

The binding of chemokines to their receptors causes a characteristic increase in cytosolic calcium, representing one of the earliest biochemical events that occur in cells in response to a ligand binding its cognate receptor (21-22). Accordingly, we hypothesized that the THP-1 cells should exhibit CXCL17-mediated Ca⁺² fluxes. As shown in FIG. 1B, we observed a strong Ca⁺² flux following addition of CXCL17 in both resting and PGE2-treated cells. In agreement with the chemotactic responses, the PGE2-treated THP-1 cells also elicited a stronger Ca⁺² flux signal (FIG. 1B).

Cellular responses to chemokines are governed by several regulatory steps. Examples of these regulatory processes include the control of both agonist and chemokine receptors synthesis or chemokine degradation (23). Additionally, there is a rapid mechanism that involves the activation of a receptor inactivation signaling pathway, know as desensitization. This phenomenon is due to the activation of a cascade of feedback inhibitors, including arrestins and G protein-coupled receptor kinases (24), and may be designed to prevent potential damaging effects of prolonged activation. Chemokine receptors therefore become desensitized for a certain period of time following activation.

We tested CXCL17-driven desensitization using THP-1 cells. As shown in FIG. 1C, CXCL17 desensitizes itself but not the Ca⁺² flux induced by CCL2, another chemokine that induces strong responses in THP-1 cells (25). Conversely, CCL2 did not desensitize CXCL17-mediated responses, indicating that these two chemokines signal through different receptors (CCL2 binds CCR2).

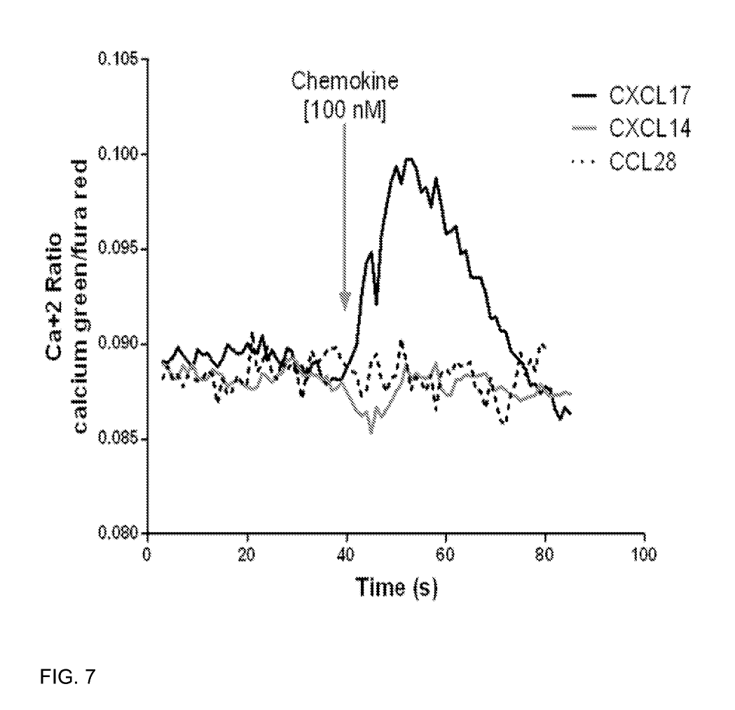

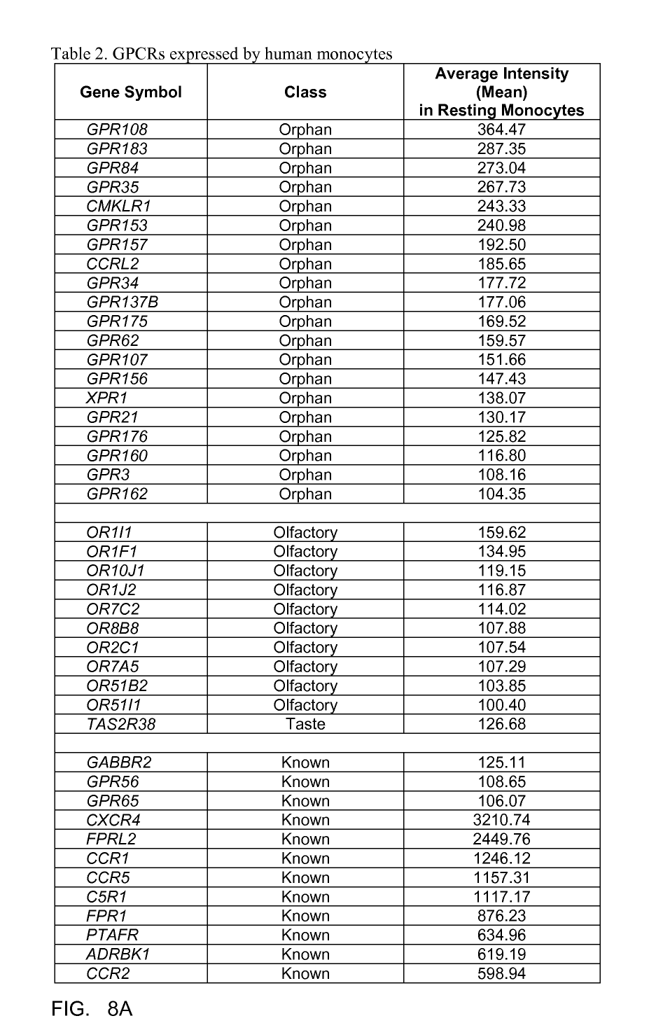

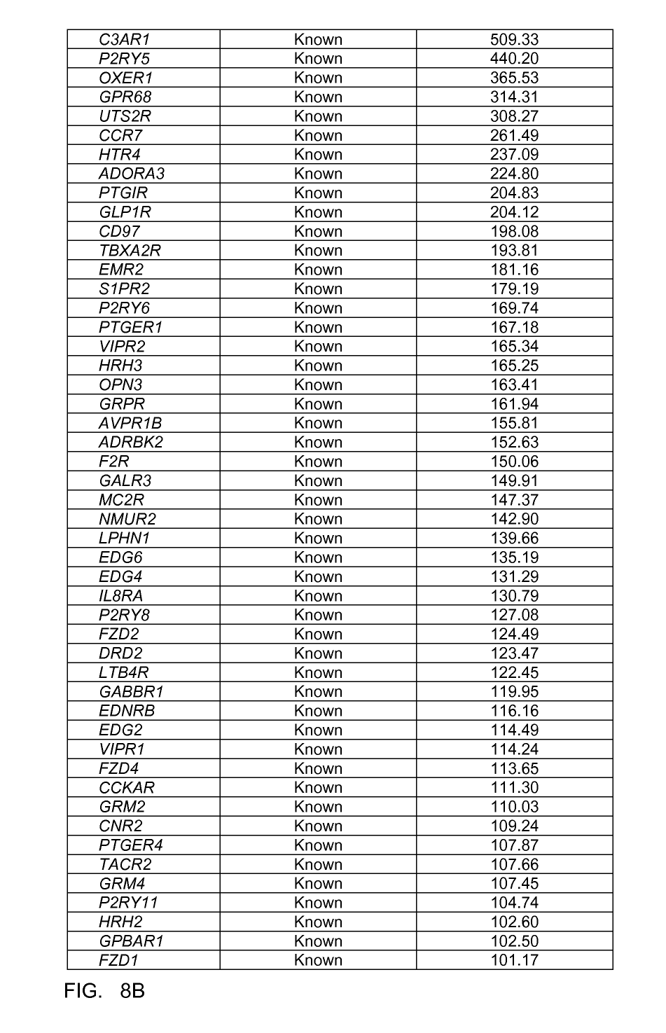

The previous results indicated that CXCL17 signals through an unidentified receptor expressed by CXCL17-responding cells. As mentioned above, CXCL17 induces chemotaxis of monocytes and dendritic cells (FIG. 1A, (2)). We therefore used a comprehensive human gene expression microarray database, a ‘Body Index of Gene Expression’ (BIGE) database (26-27), to identify GPCRs expressed by monocytes. This screen yielded close to 90 GPCRs, 10 of which were annotated as olfactory, 60 were known (annotated), and 20 as orphan (GPCRs with no known endogenous ligands) (FIG. 8, Table 2). To search for its receptor, we first tested whether CXCL17 bound or activated other known chemokine receptors including those known to be expressed by macrophages. Binding and/or signaling studies confirmed that CXCL17 does not bind or signal to CCR2, CCR5, CXCR2, CXCR3, CXCR4, CXCR7 and CCX-CKR. Furthermore, CXCL14 or CCL28 do not bind GPR35 either (FIG. 7). Therefore, we predicted that CXCL17 must bind a novel, as yet unidentified chemokine receptor. We decided to undertake experiments aimed at the identification of the CXCL17 receptor.

We focused on the orphan GPCRs and prioritized for screening those GPCRs that showed structural similarities to other chemokine receptors as well as an expression pattern similar to CXCL17. These criteria narrowed the list of candidates. We first screened CCRL2, a GPCR that exhibits many chemokine receptor like characteristics and is expressed in macrophages and DCs (28), by producing transfectants that were tested in Ca⁺² flux assays with CXCL17. In agreement with a recent report (29), we observed no calcium fluxes in response to CXCL17 (data not shown). Our next candidate was GPR35. GPR35 was first identified as a class A orphan GPCR gene (30). GPR35 is expressed in several mucosal tissues including the gastrointestinal tract (31) as well as some hematopoietic cells such as monocytes (32), basophils and eosinophils (33); and also shows relatively high expression in adult lung (34). Up-regulation of GPR35 has been found in human mast cells upon stimulation with IgE antibodies (33), human macrophages treated with benzo [a] pyrene (35) and gastric cancer cells (31).

Kynurenic acid, a tryptophan metabolite of the kynurenine pathway, 2-Acyl lysophosphatidic acid (2-acyl-LPA) and some tyrosine metabolites have been identified as agonists of GPR35 (36-37); however, whether alternative endogenous GPR35 agonists exist remains controversial.

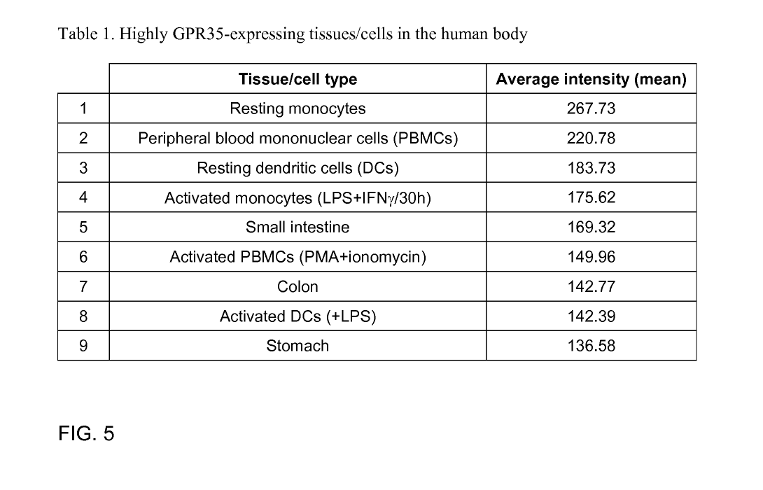

The expression of GPR35 in the BIGE database revealed that the top GPR35-expressing locations/cells include resting monocytes (FIG. 5, Table 1); as expected, resting DCs are also present in this list and show relatively high expression of GPR35 (FIG. 5, Table 1). These immune cell types show chemotaxis in response to CXCL17 ((2) and unpublished data). The receptor expression in the remaining tissues on the list is strongly mucosal and correlates with the known CXCL17 expression pattern (3).

The GPR35 gene is located on the long arm of the chromosome 2 at 2q37.3 (FIG. 2A). Interestingly, the gene encoding CXCR7 is located in a neighboring locus. This observation is interesting because phylogenetic sequence analysis indicates that CXCR7 is closely related to GPR35 (FIG. 2B). Yet, CXCL17 does not bind to CXCR7 as it does not displace ¹²⁵I-CXCL12 from CXCR7 expressing cells. Subsequent examination of the GPR35 protein sequence revealed the presence of a DRY box at the second intracellular loop (FIG. 2C). This motif, present at a corresponding position in most known functional chemokine receptors, represents the main site for G protein coupling to these transmembrane molecules (38) and is also related with the β-arrestin recruitment regulating ligand-dependent receptor internalization (39). Furthermore, we also detected the presence of a conserved Asp residue and a TxP (Thr-Xaa-Pro) motif at the second transmembrane domain. These features are highly conserved structural determinants in chemokine receptors and play an important role in receptor activation (40-41). These GPR35 structural features along with its tissue expression pattern strongly suggested that GPCR35 could be the CXCL17 receptor.

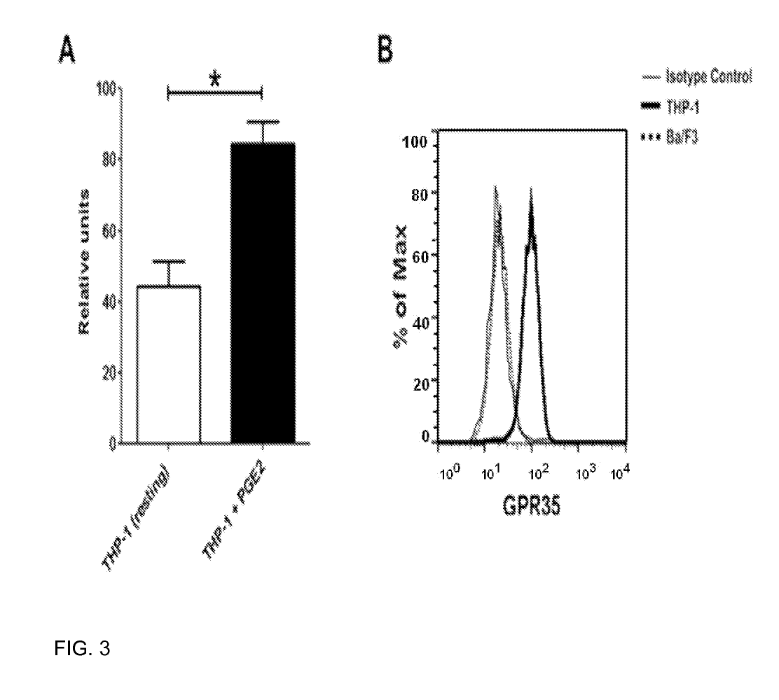

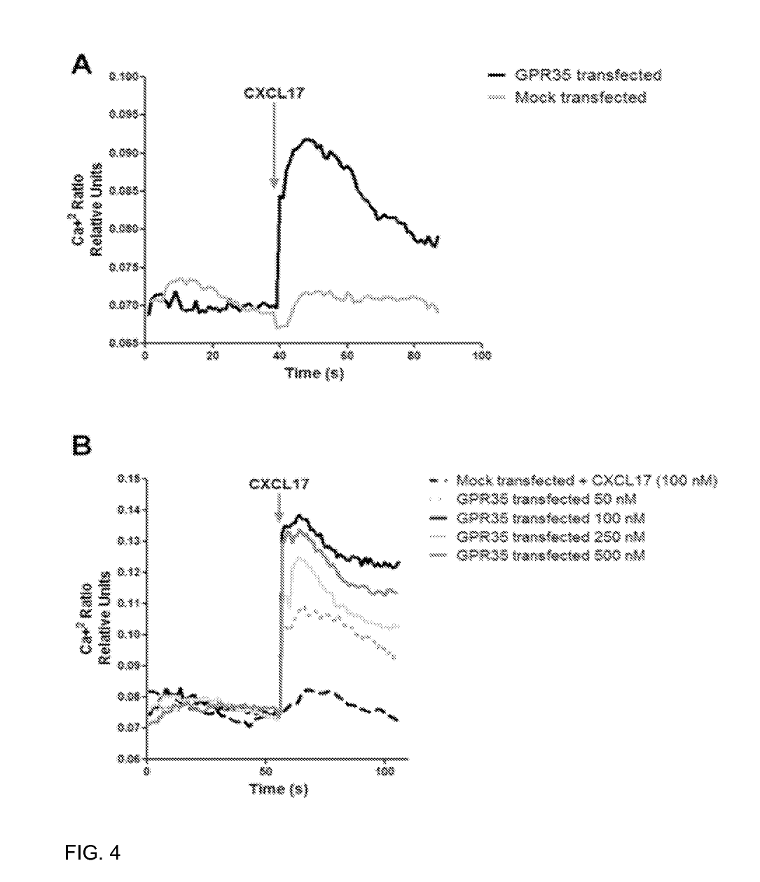

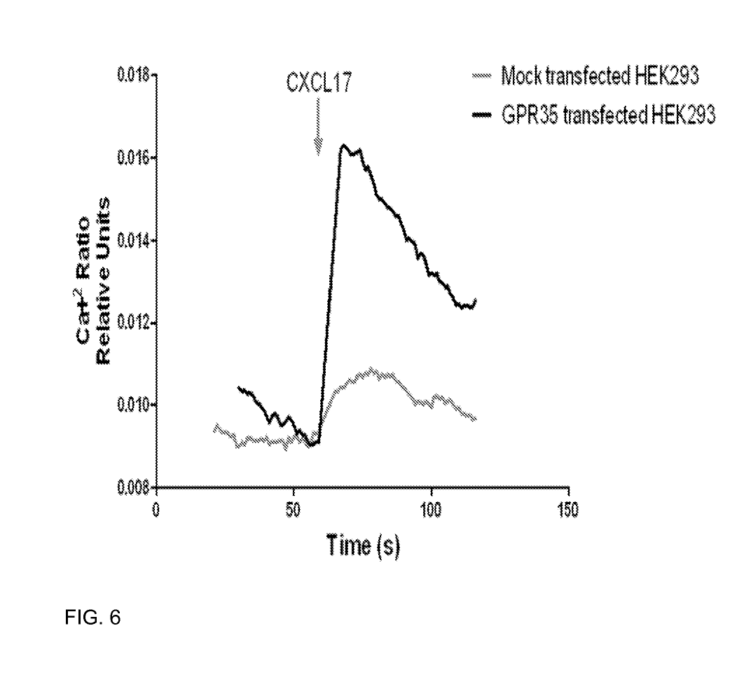

Using quantitative real-time PCR (qRT-PCR), we confirmed that GPR35 is expressed in resting THP-1 monocytes and is significantly up-regulated upon PGE2 stimulation (FIG. 3A). The expression of GPR35 in THP-1 cells was confirmed by flow cytometry (FIG. 3B). We sought to demonstrate that we could establish a calcium flux in response to CXCL17 in previously non-responsive cells by transfecting this receptor into a GPR35 null cell line. The mouse pro-B cell line Ba/F3 does not express GPR35 (42), so we used these cells for transfection experiments. When human GPR35-transfected mouse Ba/F3 cells were stimulated with recombinant human CXCL17, we observed a robust calcium flux response (FIG. 4A). Importantly, this response was not detected in mock-transfected control cells. Moreover, we also noticed a CXCL17 dose-response pattern, with increasing Ca⁺² spikes corresponding to increasing concentrations of CXCL17 (FIG. 4B). Similar results were obtained when GPR35 was transfected into other cells (FIG. 6). Additionally, we tested whether other mucosal-expressed chemokines such CXCL14 or CCL28 could induce signaling trough GPR35 without success (FIG. 7). Taken together, these observations indicate that GPR35 is a CXCL17 receptor.

CXCL17 belongs to the C-X-C chemokine sub-family and these ligands usually bind C-X-C chemokine receptors (43). Seven GPCR members compose this sub-class of chemokine receptors: CXCR1 to CXCR7 (1). Considering the ability to GPR35 to functionally respond to CXCL17, we propose to renaming GPR35 chemokine (C-X-C motif) receptor 8 (CXCR8).

The identification of CXCR8 as the CXCL17 receptor represents a important contribution to the chemokine field since the last chemokine-binding receptor (CXCR7-which binds CXCL11 and CXCL12) was reported over eight years ago (44). The physiological significance of the CXCL17/CXCR8 axis in mucosal sites remains to be explored. However, GPR35 has already been identified as a potentially important target for gastrointestinal diseases (31). Importantly, genome-wide association studies (GWAS) identified a GPR35 missense single nucleotide polymorphism strongly linked to primary sclerosing cholangitis with subsequent ulcerative colitis (5). Taken together, these observations strongly suggest that the CXCL17/CXCR8 axis is an important macrophage recruitment signal in the respiratory and digestive system, and further suggest that this axis is involved in the pathogenesis of inflammatory diseases of the gut, and given our observation of strong CXCL17 upregulation in IPF (3), also in lung pathologies. Given the importance of inflammation in both lung and gastrointestinal pathologies, we predict that the CXCL17/CXCR8 axis will be shown to be a major player in various human diseases.

### Example 2

### Cells and Reagents

Human THP-1 acute monocytic leukemia cells (American Type Culture Collection, Rockville, Md.) and an IL-3-independent clone of murine bone marrow-derived pro-B-cell line Ba/F3 (Leibniz Institute DSMZ-German Collection of Microorganisms and Cell Cultures, Braunschweig, Germany) are maintained in complete RPMI medium (10% fetal bovine serum, penicillin 1000 U/mL, streptomycin 1000 U/mL, and glutamine 20 mmol/L, everything from Corning-Cellgro, Manassas, Va.). The reagents used for the different experiments presented include: purified rabbit IgG (Jackson ImmunoResearch, West Grove, Pa.) and polyclonal rabbit anti-human GPR35 (Cayman Chemicals, Ann Arbor, Mich.). The human GPR35 clone can be obtained from The Missouri S&T cDNA Resource Center (Rolla, Mo.) consisting in the cDNA of the G protein coupled receptor 35 (GPR35) (wild type) cloned into pcDNA3.1+ expression vector (Life Technologies, Carlsbad, Calif.) at EcoRI (5′) and XhoI (3′). The open reading frame is amplified by the PCR from human genomic DNA. Insert size=930 bp. Gene bank accession number: AY275467.

### BIGE Database

The construction of the BIGE database has been described (3, 27). Briefly, tissues or cells corresponding to 105 different sites of the human body were obtained within 5 h postmortem. RNA was prepared as described and used to prepare cDNA to be hybridized to U133 2.0 gene arrays (Affymetrix, Santa Clara, Calif.). The resulting data were normalized, and a probeset corresponding to GPR35 (210264_at) was used to determine the expression of GPR35 in the human body.

### Quantitative Real-Time PCR Analysis

The quantitative real-time PCR (qRT-PCR) data are generated with a Roche Lightcycler 480 using a Universal Probe Library-based system (Roche annotation needs to go here). Briefly, total RNA is extracted from THP-1 cells using the Qiagen's RNeasy RNA purification kit. Equal concentrations of total RNA are used in a reverse transcription reaction to generate cDNA (Qiagen, Valencia, Calif.). 50 ng of each cDNA is used per 40-cycle PCR run. Gene-specific primers and corresponding Universal Probe Library are used for each reaction to quantitatively detect the amount of CXCL17 and control genes transcripts in each tissue sample. The results are processed and analyzed using GraphPad Prism software (on the World Wide Web at.graphpad.com).

### Chemotaxis Assays

Chemotaxis assays are performed using 24 well transwell migration plates (Corning, N.Y.), which contain an upper insert and lower chamber. 200 ng/mL of recombinant chemokine (R&D Systems, Minneapolis, Minn.) in 600 μl of chemotaxis buffer (C-buffer) (incomplete RPMI, Mediatech, Manassas, Va.) is added to the bottom chamber of the transwell plate. The transwell plates used in these assays had 5.0 μm sized pores (Corning, Corning, N.Y.). 0.5-1.0×10⁶ cells are used as the input number of cells for all cell lines tested unless otherwise noted. Prior to their addition to the top insert assay plate, the cells are washed three times in C-buffer. The assay is incubated at 37° C. and 5% CO₂ for 18-20 hours. Chemotaxis is periodically monitored using a microscope. Where noted, cells are treated with 200 ng/mL of pertussis toxin (PTX) (Sigma, Saint Louis, Mo.) or 10 μM prostaglandin E2 (PGE2) (Sigma) for 24 hours prior to the start of the chemotaxis assay.

### Quantification of Chemotaxis by Flow Cytometry

This protocol is adapted from Proudfoot et al. (45) Briefly, at the end of the chemotaxis assay, the chemotaxed cells are collected from the bottom chamber of the plate, spun down in FACS tubes, and resuspended in 200 μL of 1×PBS. Standards can be generated by making 10-fold dilutions of cells ranging from 1.0×10⁶ to 1.0×10² cells in 200 μL of 1×PBS. The cell counts for the standards and all of the chemotaxed cells are recorded as the number of events counted in 30 seconds. Since the precise number of cells is known for the standards, their cell counts are used to generate a standard curve. The trendline and equation resulting from this standard curve is used to calculate the relative number of cells that chemotaxed for each cell line or primary cell analyzed. A FACSCalibur machine (Becton Dickinson, Franklin Lakes, N.J.) is used for these quantification experiments.

### GPR35 Transfection Asssays

Ba/F3 cells are resuspended in cytomix buffer (120 mM KCl, 0.15 mM CaCl₂, 25 mM HEPES/KOH, pH 7.6, 2 mM EGTA, 5 mM MgCl₂) at a final density of 2×10⁷ cells/mL transferring 500 μL of suspension to a 0.4 cm electroporation cuvette (USA Scientific, Ocala, Fla.). Then, twenty μg of pcDNA3.1+/GPR35 DNA is transfected into the cells. Plasmid DNA is added to the cell suspension in the cuvette and mixed by gentle pipetting. The mixture is then exposed to a single electric pulse of 300 V with a capacitance of 960 μF using a Bio-Rad (Hercules, Calif.) pulse system. The cells are allowed to recover in complete culture medium at 37° C. (5% CO₂ atmosphere) for 48 h before harvesting and performing Ca⁺² mobilization assays.

### Calcium Mobilization Assays

For calcium studies in THP-1 or transfected Ba/F3 cells, 5×10⁷ cells/mL are loaded with calcium green-1-AM and fura-red-AM (Life Technologies, Carlsbad, Calif.) both at a final dye 10 μmol/L concentration for 30 minutes at 37° C. in non-supplemented RPMI 1640. Loaded cells are washed once in Hanks balanced salt solution containing 0.14 g/L of CaCl₂ (HBSS, Corning-Cellgro), resuspended at 1.5×10⁶ cells/mL in HBSS, and immediately placed on ice protecting them from light. Prior to activation, cells are warmed to 37° C. for 15 minutes. Following 30 seconds of data acquisition, cells are stimulated by addition of different amounts of human recombinant CXCL17 (R&D Systems, Minneapolis, Minn.), using the stimulation with 100 μM Ionomycin (Sigma, Saint Louis, Mo.) at a final stage to determine the viability of every cell-group analyzed, representing a positive control-stimulus. The calcium green versus fura red fluorescence ratio of individual cells is measured by means of a FACSCalibur flow cytometer (Becton Dickinson) before and after the addition of activators and analyzed by means of the FlowJo FACS software (Tree Star Inc.). Data are presented in arbitrary units as a function of fluorescence (relative intracellular calcium) versus time.

### Example 3

The term “epitope” means a protein determinant capable of specific binding to an antibody or a binding domain such as one or more loops of a scaffold-based or receptor proteins.

These epitopes usually consist of chemically active surface groupings of molecules such as amino acids or sugar side chains and usually have specific three-dimensional structural characteristics, as well as specific charge characteristics. Conformational and nonconformational epitopes are distinguished in that the binding to the former but not the latter is lost in the presence of denaturing solvents or heat treatment.

The conformational epitopes result from conformational folding of the target molecule, which arise when amino acids from differing portions of the linear sequence of the target molecule come together in close proximity in 3-dimensional space.

Chemokines share a conserved 3D structure, the so-called IL8-like chemokine fold, which is stabilized by cysteine residues forming intra-molecular disulfide bonds.

Interestingly, the predicted IL8-like chemokine structure of CXCL17 reveals disulfide bonds in non-canonical regions in 3D structure while still maintaining an active fold. The low sequence similarity to other known members of the family and its cysteine patterns differing from those in known chemokines are the reasons why chemokine CXCL17 escaped annotation by standard sequence-based methods (2).

Chemokine receptor activation involves interactions between chemokine N-loop and receptor N-terminal residues, and between chemokine N-terminal and receptor extracellular/transmembrane residues (46), demonstrating that the conformational state of this interaction is critical.

So, by heating a recombinant CXCL17 preparation (95° C., 10 minutes) followed by an immediate heat-shock (4° C., 5 min) we can denature and prevent the renaturation of chemokine into active conformation. By doing this, the native 3-dimensional conformation of the protein is destroyed but the protein primary structure, as defined by the amino acid sequence, should remain intact. The intact polypeptide can be evaluated by SDS-PAGE, or other analytical method, to establish that the polypeptide and sequence remain intact.

When the native “conformational active” CXCL17 is added to cells transfected with CXCR8, that were previously loaded with Ca+2 sensitive dyes (Fura Red plus Calcium Green), an increase in intracellular Ca+2 concentration as measured by an increase in fluorescence ratio can be detected by a flow cytometer. If the heat-denatured CXCL17 is added in the same assay, the cells are not responsive, as indicated by the absence of increased Ca+2 signaling. This response demonstrates that the polypeptidic sequence by itself of CXCL17 is not responsible for binding and functional activating CXCR8 but its conformational-integral native form is.

### Example 4

The CXCL17/CXCR8 interaction is likely to play a major role in gastrointestinal inflammatory disorders. (31). Importantly, genome-wide association studies (GWAS) identified a CXCR8/GPR35 missense single nucleotide polymorphism strongly linked to primary sclerosing cholangitis with subsequent ulcerative colitis (5). This kind of information makes an involvement of CXCL17/CXCR8 in gastrointestinal inflammatory disorders very likely. The effectiveness of agonists and/or antagonists of the CXCL17/CXCR8 interaction can be assayed using pre-clinical mouse models of gastrointestinal disorders. The two predominant murine model of colitis are induced using dextran sodium sulfate (DSS) (47-48), or 2,4,6-trinitro benzene sulfonic acid (TNBS) (49-51). The DSS model imitates human colitis more than the TNBS model because it can be induced in either an acute or a chronic form (47-49).

These models are well established and have been shown to yield consistently reproducible results. Therefore, the effect of adding of agonists or antagonists to these systems should be easy to detect in the assay readout when the responses of agonist/antagonist treated and untreated mice are compared upon selected pharmacological dosing in the therapeutic range. Specifically, disease pathogenesis and severity would be compared between the two cohorts of animals. The ideal administered dose and route of delivery of the agonists/antagonists could also be easily varied, tested and ultimately determined using these models.

CXCL17/CXCR8 deficient (knockout) mouse strains can also be used to predict the efficacy of antagonists in gastrointestinal disorders. These mouse strains are lacking expression of either the ligand or receptor, and therefore will behave similar to wild type (WT) mice treated with an antagonist. The response of CXCL17/CXCR8 deficient mice to the pre-clinical murine models of colitis can be compared to that of WT mice, and conclusions about the efficacy of the specific antagonists can be made. The animal models may also be used to establish whether the chemokine or receptor evaluation may provide diagnostic or therapeutic subsetting of specific animals to determine dosing and therapeutic strategy.

In one example, a monoclonal antibody targeted against CXCL17 is used in the acute murine DSS model of colitis (52). The antibody is selected to confirm that it inhibits the CXCL17/CXCR8 interaction by inhibiting the calcium flux observed in a BA/F3 cell transfected with CXCR8 as shown in the drawings. The experiment can use four cohorts of mice, e.g., one cohort that receives isotype control antibody, one cohort that receives the anti-CXCL17 antibody, one cohort that receives isotype control antibody and DSS, and a final cohort that receives anti-CXCL17 antibody and DSS. Over the experimental period, the mice receiving DSS are dosed in their drinking water at Day 1 and Day 5; control mice are just given autoclaved drinking water. The anti-CXCL17 antibody or isotype control antibody are given at the appropriate therapeutic dose to the mice through intraperitoneal (i.p.) or intravenous (i.v.) injection at three different times during the DSS treatment: one injection before starting DSS treatment and two injections during DSS treatment.

The efficacy of the anti-CXCL17 antibody can be assayed by analyzing the changes in weight of the mice and the development of gastrointestinal symptoms, e.g., diarrhea/bloody stools, during the course of the DSS treatment (52). The levels of inflammation of the colon are analyzed at the end of the experiment, e.g., using Q-PCR, immunohistochemistry (IHC) and/or immunophenotyping of individual immune cell populations (52).

The example can be used in other subjects, including humans, that may have gastrointestinal diseases such as Crohn's disease, ulcerative colitis, celiac disease, or others. See, e.g., Hauser, S. C. Mayo Clinic Gastroenterology and Hepatology Board Review, Fourth Ed. Mayo Clinic Scientific Press, 2013; Hawkey et al., Clinical and Gastroenterology and Hepatology, Second Ed. Wiley-Blackwell, 2012; and Yamada T. et al. Yamada's Handbook of Gastroenterology, 3^{rd} Ed. Wiley-Blackwell, 2013. Genetic models, e.g., knock-out animals, may be particularly useful test subjects for therapeutic testing.

### Example 5

The efficacy of agonists or antagonists in targeting of the CXCL17/CXCR8 interaction can be shown using pre-clinical murine models of respiratory disease. The murine bleomycin model of human idiopathic pulmonary fibrosis (IPF) is widely used to study IPF in animals (53-55). See, e.g., Models of Lung Disease, edited by Joan Gil, copyright 1990; and Fishman's Pulmonary Diseases and Disorders, Fishman et al, copyright 2008.

Using two cohorts of animals (agonist/antagonist treated versus untreated), the disease progression and severity in both cohorts are compared. Appropriate therapeutic dosing and therapeutic treatment are applied to the animals. Conclusions about the effectiveness of the agonists/antagonists are made after analyzing the results of these experiments. Additionally, the amount of agonist/antagonist and route of delivery are compared using this model.

CXCL17/CXCR8 deficient (knockout) mouse strains are used to predict the efficacy of antagonists in respiratory disorders. These mouse strains lack expression of either the ligand or receptor, and therefore will behave similar to wild type (WT) mice treated with an antagonist. The response of CXCL17/CXCR8 deficient mice to the pre-clinical murine models of IPF are compared to that of WT mice, and conclusions about the efficacy of the specific antagonists are made.

One example uses a monoclonal antibody targeted against CXCR8 as a ligand antagonist in a murine bleomycin model of IPF. The antibody is tested to confirm that it inhibits the CXCL17/CXCR8 interaction by inhibiting the calcium flux observed in a BA/F3 cell transfected with CXCR8 as shown in the drawings. The experiment may use, e.g., four cohorts of mice: one cohort that receives isotype control antibody, one cohort that receives the anti-CXCR8 antibody, one cohort that receives isotype control antibody and bleomycin, and a final cohort that receives anti-CXCR8 antibody and bleomycin. Over the experimental period, the mice receiving bleomycin are given doses, e.g., through intraperitoneal (i.p.) or intratracheal (i.t.) instillation (22294226). To achieve fibrosis of the lungs, bleomycin is dosed multiple times over a 2-3 week period, after which fibrosis of the lungs is evaluated. The anti-CXCR8 antibody or isotype control antibody are given to the mice, e.g., through intraperitoneal (i.p.) or intravenous (i.v.) injection three different times during the bleomycin treatment: one injection before starting bleomycin treatment and two injections during bleomycin treatment.

The efficacy of the anti-CXCR8 antibody is assayed, e.g., by analyzing the changes in weight of the mice during the course of the DSS treatment (56). Inflammation of the lungs is analyzed at the end of the experiment, e.g., by measuring collagen and/or hydroxyproline content of the lungs and/or immunohistochemistry (IHC) of the lung (56).

An analogous example is applicable to other subjects including humans affected, for example, with idiopathic pulmonary fibrosis or other respiratory ailments. See, e.g., Judd, S, J, Respiratory Disorders Sourcebook, 2^{nd} Ed. Health Reference Series, 2012; and Lechner, A. Respiratory, An integrated approach to disease; McGrawHill LANGE, 2012.

### Example 6

Multiple sclerosis (MS) is an immune-mediated demyelinating disease of the human central nervous system (CNS) that is the most common non-traumatic cause of disability in young adults. See Holland, N. et al Multiple Sclerosis, 4^{th} Ed demos Health 2012). It is characterized by the activation and recruitment of T cells and macrophages to the lesion site, and the production of demyelinating antibodies (57).

Experimental autoimmune encephalomyelitis (EAE) is an animal model for MS (58). It is based on the induction of an autoimmune response to injected myelin proteins such as proteolipid protein, myelin basic protein, and myelin oligodendrocyte glycoprotein in mice and rats.

EAE can also be induced by passive transfer of T cells specific for myelin antigens. Using various immunization protocols, acute and chronic-relapsing EAE models can be induced.

The role of different chemokines and chemokine receptors in the pathogenesis of EAE has been extensively investigated. CCL1, CCL2, CCL3, CCL4, CCL5, CCL7, CXCL1, CXCL9, CXCL10, CXCL11, and CXCL16 chemokines were reported to be expressed in the CNS in EAE models. CCL2 chemokine (monocytes chemoattractant protein-1) acts on monocytes, activated T cells, natural killer (NK) cells, and microglia by binding to the CCR2 receptor. CCL2 can be produced by astrocytes, microglia, endothelial cells, and macrophages. Interestingly, CCL2-deficient mice were markedly resistant to the induction of EAE, and showed a significant reduction in macrophage recruitment into the CNS (59). Furthermore, CCR2 knockout mice did not develop clinical signs of the disease, and the upregulation of both the CCL2 chemokine and CCR2 receptor in the CNS was associated with a relapse of EAE (60-61).

Results also show that CCR1 knockout mice can develop an attenuated form of the disease (62). Among CCR1 ligands there are CCL3 (MIP1-a, macrophage inflammatory protein-1), and CCL5 (RANTES, regulated upon activation, normal T cell expressed and secreted), the chemokines which are expressed in the CNS lesions in EAE. It was found that treatment with anti-CCL3 antibodies inhibited EAE onset and reduced the accumulation of mononuclear cells in the CNS (63).

As CXCL17/CXCR8 axis is a major chemotactic determinant for monocytes/macrophages, the treatment of MS should include a therapy to block either the chemokine or the receptor-induced recruitment of these cells to the CNS. This blocking agent (antagonist) in this example is a CXCL17 mutein that is capable of binding CXCR8 but does not signal. This is shown by its ability to block the calcium flux induced by CXCL17 in CXCR8 induced by native (non mutated) CXCL17. It is also shown that CXCL17 mutein does not induce a calcium flux in the CXCR8 transfectants. Mice receive the myelin basic protein in adjuvant to induce an immune response against it and trigger experimental allergic encephalomyelitis in the animals. A control group receives placebo and the experimental group receives the CXCL17 mutein. The effect of the CXCL17 mutein is evaluated, e.g., by following the progression of EAE in the mice receiving placebo or CXCL17 mutein. The administration of the mutein to the experimental mice reduces the progression of the EAE.

### Example 7

Another use of embodiments of this invention is to identify compounds that either antagonize the CXCL17/CXCR8 interaction or mimic CXCL17 (are agonists of CXCR8). To this end, it is possible to use technologies like the FLIPR described above to screen chemical compound libraries for compounds that will block the ability of CXCL17 to induce a calcium flux in CXCR8 transfectants. In an alternate strategy, the CXCR8 transfectants can be used to identify compounds that induce calcium fluxes in these transfectants but not in corresponding untransfected cells. The latter compounds would be agonists of CXCR8.

In another embodiment, the invention can also be used to identify antibodies that block the CXCL17/CXCR8 interaction. To this end, antibodies can be directed either against the ligand (CXCL17) or against the receptor (CXCR8). However we can predict that only a subset of antibodies directed against these proteins will be able to block the ability of CXCL17 to signal through CXCR8. To identify these blocking antibodies, we can test them for their ability to inhibit calcium fluxes induced by CXCL17 in CXCR8 transfectants. To do this we would place CXCR8 transfectants with the antibodies to be tested, and then add CXCL17 to induce a calcium flux that is detectable by various instruments (fluorimeter, fluorescence activated cell sorter, etc). Those antibodies that inhibit calcium fluxes represent blocking antibodies (CXCL17/CXCR8 antagonists). The antibodies can be produced from immunized animals (mice, rats, hamsters, rabbits) with either CXCL17 or with CXCR8 transfectants. Once a titer is detected, the spleen can be used to either fuse to a myeloma cell partner in order to produce hybridomas or a phage display library can be produced. Either technique can lead to the identification of antibodies that bind either CXCL17 or CXCR8 and their ability to block signaling through CXCR8 can be measured by inhibition of calcium flux.

### Example 8

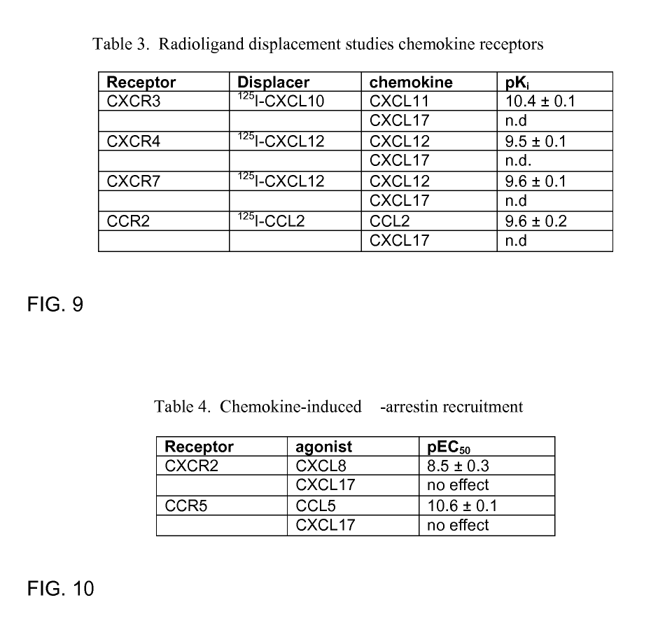

Radioligand displacement studies of various chemotaxis receptors were carried out. For the radioligand binding assay, membranes from HEK293T cells transiently expressing the respective chemokine receptors were incubated with ˜50 pmol ¹²⁵I-chemokine and increasing concentrations of chemokine (control) or CXCL17. Cells were incubated for 3 h at 4° C. and washed twice with binding buffer containing 0.5 M NaCl. After harvesting the samples with lysis buffer, the remaining cell-bound radioactivity was counted. The results in FIG. 9 (Table 3) show that CXCL17 did not displace various ligands from their respective receptors (n.d.=not detectable).

β-arrestin recruitment assay were carried out using PathHunter™ CCR5 or CXCR2 expressing β-arrestin cells (DiscoveRx (Fremont, Calif.)) to monitor chemokine-induced β-arrestin recruitment based on enzyme complementation. The results in FIG. 10 (Table 4) show that CXCL17 had no effect on the cells.

### Example 9

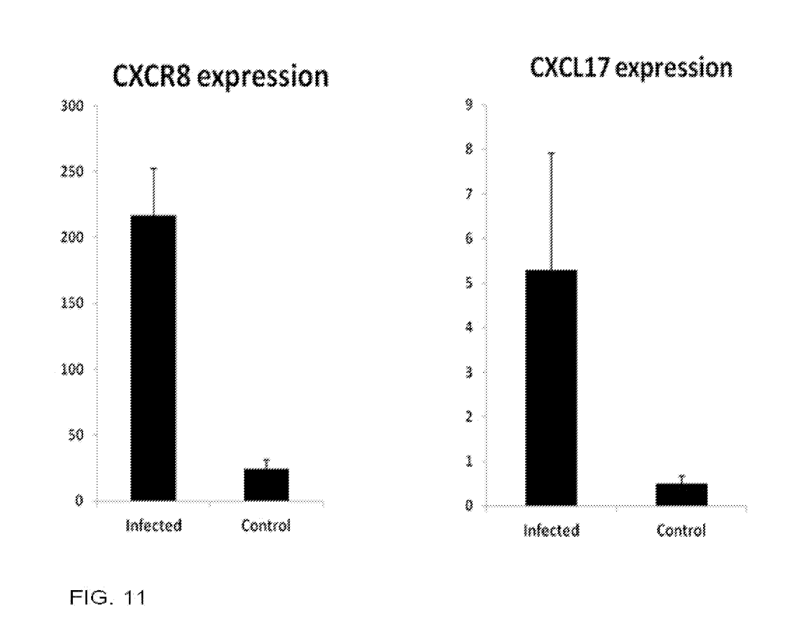

The expression of CXCR8 and CXCL17 in Salmonella-infected mice was determined. Small intestines from wild type C57BL/6 mice infected with Salmonella were collected at the end of the experiment (1 week). RNA was extracted from each intestine for gene expression analysis by RT-qPCR. As shown in FIG. 11, CXCR8 and CXCL17 expression is elevated in Salmonella infected mice compared to mock infected mice. These results indicate that the expression of both CXCR8 and CXCL17 are induced in the intestine upon inflammatory conditions, supporting a role for the CXCR8/CXCL17 in gut inflammation.

### Example 10

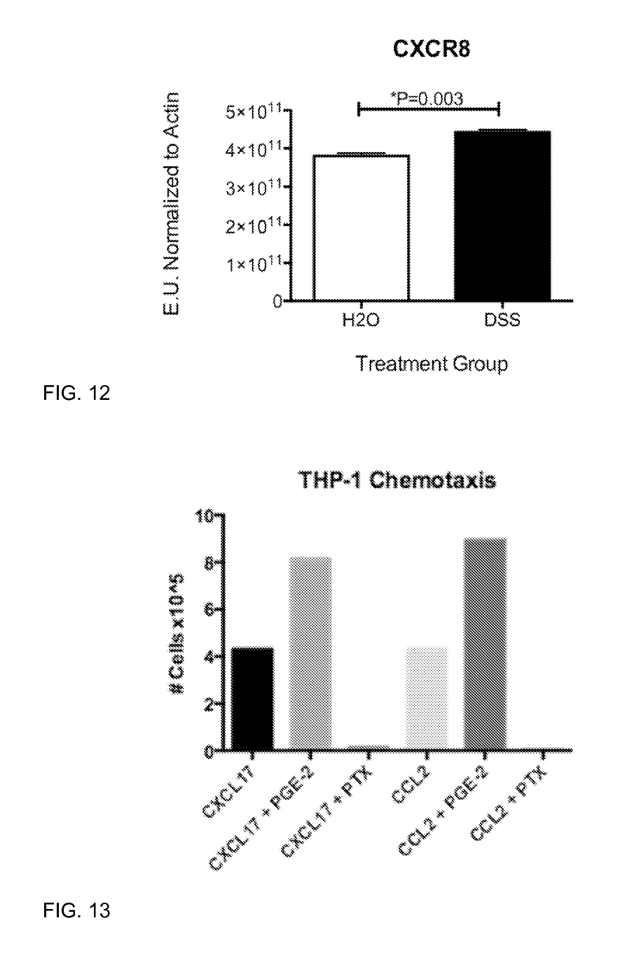

CXCR8 levels were studied in a mouse model of ulcerative colitis. Dextran Sodium Sulfate (DSS) was used to induce gut inflammation as a of model Ulcerative Colitis (UC) in wild type (C57Bl/6) mice. After 7 days of treatment colons were collected from DSS treated and mock treated mice for gene expression analysis. As shown in FIG. 12, expression of CXCR8 is elevated in DSS treated mice compared to H₂O treated mice. These results support the role of the CXCR8/CXCL17 axis in the pathogenesis of inflammatory diseases of the gut.

### Example 11

The chemotactic activity of CXCR8:CXCL17 was compared to that of CCR2:CCL2, an established and well-characterized macrophage chemoattractant axis. 1×10̂6 THP-1 cells were loaded into the top chamber of a transwell chemotaxis plate with 100 ng of recombinant human chemokine loaded in the bottom chamber. After 20 hours chemotaxis was measured by counting the cells that migrated into the bottom chamber. Pertussis toxin (PTX) was used to inhibit the chemotactic response and confirm that it involves G-protein signaling. Prostaglandin-E2 (PGE₂) enhances chemotaxis to both CXCL17 and CCL2. As shown in FIG. 13, chemotaxis mediated by CXCR8:CXCL17 was comparable to CCL2.

### Example 12

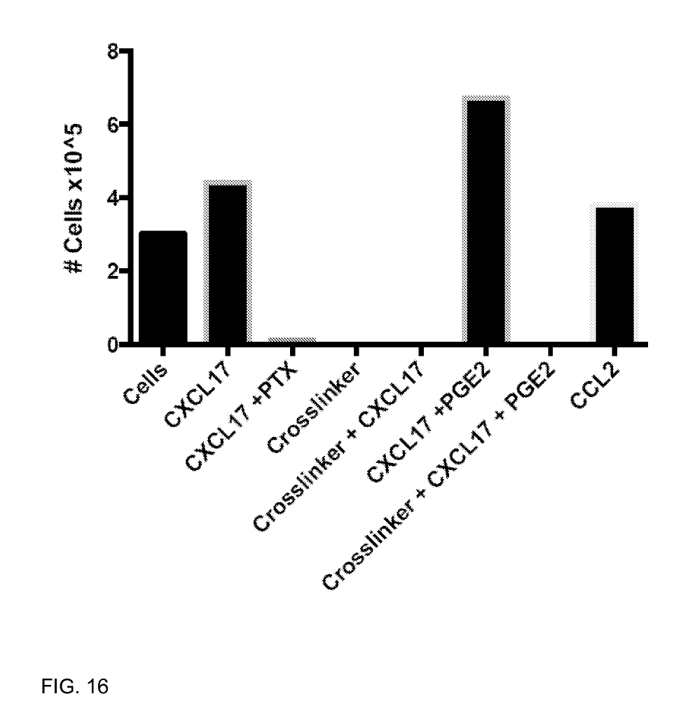

THP-1 cells were analyzed for their chemotactic response to recombinant human CXCL17 using transwell migration assays. Cells were tested alone, after 24 pre-treatment with Prostaglandin E2 (PGE2), pertussis toxin (PTX) or after treatment with glutaraldehyde. PGE2 amplifies the responsiveness of THP-1 cells to CXCL17. PTX blocks signaling through chemokine receptors (Gαi G-Coupled Protein Receptors (GPCRs)), so THP-1 cells are unable to chemotax in response to CXCL17. 0.05% glutaraldehyde was used to crosslink all membrane proteins of the THP-1 cells, which abolished their chemotactic ability without reducing cell viability. CCL2 was used as a positive control. 200 ng/ml of chemokine was used to induce chemotaxis. The results of crosslinking are shown in FIG. 16. The results indicate that mild crosslinking of membrane proteins eliminates chemotaxis to CXCL17.

### REFERENCES

The following publications are incorporated by reference herein:
1. Zlotnik, A., and O. Yoshie. 2012. The chemokine superfamily revisited. Immunity 36:705-716.
2. Pisabarro, M. T., B. Leung, M. Kwong, R. Corpuz, G. D. Frantz, N. Chiang, R. Vandlen, L. J. Diehl, N. Skelton, H. S. Kim, D. Eaton, and K. N. Schmidt. 2006. Cutting edge: novel human dendritic cell- and monocyte-attracting chemokine-like protein identified by fold recognition methods. J Immunol 176:2069-2073.
3. Burkhardt, A. M., K. P. Tai, J. P. Flores-Guiterrez, N. Vilches-Cisneros, K. Kamdar, O. Barbosa-Quintana, R. Valle-Rios, P. A. Hevezi, J. Zuniga, M. Selman, A. J. Ouellette, and A. Zlotnik. 2012. CXCL17 is a mucosal chemokine elevated in idiopathic pulmonary fibrosis that exhibits broad antimicrobial activity. J Immunol 188:6399-6406.
4. Sharma, M. 2010. Chemokines and their receptors: orchestrating a fine balance between health and disease. Crit Rev Biotechnol 30:1-22.
5. Ellinghaus, D., T. Folseraas, K. Holm, E. Ellinghaus, E. Melum, T. Balschun, J. K. Laerdahl, A. Shiryaev, D. N. Gotthardt, T. J. Weismuller, C. Schramm, M. Wittig, A. Bergquist, E. Bjornsson, H. U. Marschall, M. Vatn, A. Teufel, C. Rust, C. Gieger, H. E. Wichmann, H. Runz, M. Sterneck, C. Rupp, F. Braun, R. K. Weersma, C. Wijmenga, C. Y. Ponsioen, C. G. Mathew, P. Rutgeerts, S. Vermeire, E. Schrumpf, J. R. Hov, M. P. Manns, K. M. Boberg, S. Schreiber, A. Franke, and T. H. Karlsen. 2012. Genome-wide association analysis in Primary sclerosing cholangitis and ulcerative colitis identifies risk loci at GPR35 and TCF4. Hepatology.
6. Herbst, R. S., J. V. Heymach, and S. M. Lippman. 2008. Lung cancer. N Engl J Med 359:1367-1380.
7. Cooper, S., and S. G. Spiro. 2006. Small cell lung cancer: treatment review. Respirology 11:241-248.
8. Molina, J. R., P. Yang, S. D. Cassivi, S. E. Schild, and A. A. Adjei. 2008. Non-small cell lung cancer: epidemiology, risk factors, treatment, and survivorship. Mayo Clin Proc 83:584-594.
9. Remon, J., P. Lianes, S. Martinez, M. Velasco, R. Querol, and M. Zanui. 2013. Malignant mesothelioma: new insights into a rare disease. Cancer Treat Rev 39:584-591.
10. King, T. E., Jr., A. Pardo, and M. Selman. 2011. Idiopathic pulmonary fibrosis. Lancet 378:1949-1961.
11. Selman, M., A. Pardo, and T. E. King, Jr. 2012. Hypersensitivity pneumonitis: insights in diagnosis and pathobiology. Am J Respir Crit Care Med 186:314-324.
12. Cosio, M. G., M. Saetta, and A. Agusti. 2009. Immunologic aspects of chronic obstructive pulmonary disease. N Engl J Med 360:2445-2454.
13. Jobe, A. H. 2011. The new bronchopulmonary dysplasia. Curr Opin Pediatr 23:167-172.
14. Kudo, M., Y. Ishigatsubo, and I. Aoki. 2013. Pathology of asthma. Front Microbiol 4:263.
15. White, G. E., A. J. Iqbal, and D. R. Greaves. 2013. CC chemokine receptors and chronic inflammation—therapeutic opportunities and pharmacological challenges. Pharmacol Rev 65:47-89.
16. Sun, L., and R. D. Ye. 2012. Role of G protein-coupled receptors in inflammation. Acta Pharmacol Sin 33:342-350.
17. Vetter, I. 2012. Development and optimization of FLIPR high throughput calcium assays for ion channels and GPCRs. Adv Exp Med Biol 740:45-82.
18. Qin, Z. 2012. The use of THP-1 cells as a model for mimicking the function and regulation of monocytes and macrophages in the vasculature. Atherosclerosis 221:2-11.
19. Kurth, I., K. Willimann, P. Schaerli, T. Hunziker, I. Clark-Lewis, and B. Moser. 2001. Monocyte selectivity and tissue localization suggests a role for breast and kidney-expressed chemokine (BRAK) in macrophage development. J Exp Med 194:855-861.
20. Burns, D. L. 1988. Subunit structure and enzymic activity of pertussis toxin. Microbiol Sci 5:285-287.
21. Murphy, P. M. 1996. Chemokine receptors: structure, function and role in microbial pathogenesis. Cytokine Growth Factor Rev 7:47-64.
22. Bokoch, G. M. 1995. Chemoattractant signaling and leukocyte activation. Blood 86:1649-1660.
23. Steele, A. D., I. Szabo, F. Bednar, and T. J. Rogers. 2002. Interactions between opioid and chemokine receptors: heterologous desensitization. Cytokine Growth Factor Rev 13:209-222.
24. Kohout, T. A., and R. J. Lefkowitz. 2003. Regulation of G protein-coupled receptor kinases and arrestins during receptor desensitization. Mol Pharmacol 63:9-18.
25. Gouwy, M., S. Struyf, S. Noppen, E. Schutyser, J. Y. Springael, M. Parmentier, P. Proost, and J. Van Damme. 2008. Synergy between coproduced CC and CXC chemokines in monocyte chemotaxis through receptor-mediated events. Mol Pharmacol 74:485-495.
26. Roth, R. B., P. Hevezi, J. Lee, D. Willhite, S. M. Lechner, A. C. Foster, and A. Zlotnik. 2006. Gene expression analyses reveal molecular relationships among 20 regions of the human CNS. Neurogenetics 7:67-80.
27. Lee, J., A. Hever, D. Willhite, A. Zlotnik, and P. Hevezi. 2005. Effects of RNA degradation on gene expression analysis of human postmortem tissues. FASEB J 19:1356-1358.
28. Otero, K., A. Vecchi, E. Hirsch, J. Kearley, W. Vermi, A. Del Prete, S. Gonzalvo-Feo, C. Garlanda, O. Azzolino, L. Salogni, C. M. Lloyd, F. Facchetti, A. Mantovani, and S. Sozzani. 2010. Nonredundant role of CCRL2 in lung dendritic cell trafficking Blood 116:2942-2949.
29. Lee, W. Y., C. J. Wang, T. Y. Lin, C. L. Hsiao, and C. W. Luo. 2013. CXCL17, an orphan chemokine, acts as a novel angiogenic and anti-inflammatory factor. Am J Physiol Endocrinol Metab 304:E32-40.
30. O'Dowd, B. F., T. Nguyen, A. Marchese, R. Cheng, K. R. Lynch, H. H. Heng, L. F. Kolakowski, Jr., and S. R. George. 1998. Discovery of three novel G-protein-coupled receptor genes. Genomics 47:310-313.
31. Okumura, S., H. Baba, T. Kumada, K. Nanmoku, H. Nakajima, Y. Nakane, K. Hioki, and K. Ikenaka. 2004. Cloning of a G-protein-coupled receptor that shows an activity to transform NIH3T3 cells and is expressed in gastric cancer cells. Cancer Sci 95:131-135.
32. Barth, M. C., N. Ahluwalia, T. J. Anderson, G. J. Hardy, S. Sinha, J. A. Alvarez-Cardona, I. E. Pruitt, E. P. Rhee, R. A. Colvin, and R. E. Gerszten. 2009. Kynurenic acid triggers firm arrest of leukocytes to vascular endothelium under flow conditions. J Biol Chem 284:19189-19195.
33. Yang, Y., J. Y. Lu, X. Wu, S. Summer, J. Whoriskey, C. Saris, and J. D. Reagan. 2010. G-protein-coupled receptor 35 is a target of the asthma drugs cromolyn disodium and nedocromil sodium. Pharmacology 86:1-5.
34. Horikawa, Y., N. Oda, N. J. Cox, X. Li, M. Orho-Melander, M. Hara, Y. Hinokio, T. H. Lindner, H. Mashima, P. E. Schwarz, L. del Bosque-Plata, Y. Oda, I. Yoshiuchi, S. Colilla, K. S. Polonsky, S. Wei, P. Concannon, N. Iwasaki, J. Schulze, L. J. Baier, C. Bogardus, L. Groop, E. Boerwinkle, C. L. Hanis, and G. I. Bell. 2000. Genetic variation in the gene encoding calpain-10 is associated with type 2 diabetes mellitus. Nat Genet 26:163-175.
35. Sparfel, L., M. L. Pinel-Marie, M. Boize, S. Koscielny, S. Desmots, A. Pery, and O. Fardel. 2010. Transcriptional signature of human macrophages exposed to the environmental contaminant benzo(a)pyrene. Toxicol Sci 114:247-259.
36. Milligan, G. 2011. Orthologue selectivity and ligand bias: translating the pharmacology of GPR35. Trends Pharmacol Sci 32:317-325.
37. Deng, H., H. Hu, and Y. Fang. 2012. Multiple tyrosine metabolites are GPR35 agonists. Sci Rep 2:373.
38. Daiyasu, H., W. Nemoto, and H. Toh. 2012. Evolutionary Analysis of Functional Divergence among Chemokine Receptors, Decoy Receptors, and Viral Receptors. Front Microbiol 3:264.
39. Kim, K. M., and M. G. Caron. 2008. Complementary roles of the DRY motif and C-terminus tail of GPCRS for G protein coupling and beta-arrestin interaction. Biochem Biophys Res Commun 366:42-47.
40. Govaerts, C., C. Blanpain, X. Deupi, S. Ballet, J. A. Ballesteros, S. J. Wodak, G. Vassart, L. Pardo, and M. Parmentier. 2001. The TXP motif in the second transmembrane helix of CCR5. A structural determinant of chemokine-induced activation. J Biol Chem 276:13217-13225.
41. Cancellieri, C., N. Caronni, A. Vacchini, B. Savino, E. M. Borroni, M. Locati, and R. Bonecchi. 2013. Review: Structure-function and biological properties of the atypical chemokine receptor D6. Mol Immunol 55:87-93.
42. Wu, C., C. Orozco, J. Boyer, M. Leglise, J. Goodale, S. Batalov, C. L. Hodge, J. Haase, J. Janes, J. W. Huss, 3rd, and A. I. Su. 2009. BioGPS: an extensible and customizable portal for querying and organizing gene annotation resources. Genome Biol 10:R130.
43. Murphy, P. M., M. Baggiolini, I. F. Charo, C. A. Hebert, R. Horuk, K. Matsushima, L. H. Miller, J. J. Oppenheim, and C. A. Power. 2000. International union of pharmacology. XXII. Nomenclature for chemokine receptors. Pharmacol Rev 52:145-176.
44. Han, D. K., S. Y. Jeong, Y. H. Kim, and B. G. Min. 1992. Surface characteristics and blood compatibility of polyurethanes grafted by perfluoroalkyl chains. J Biomater Sci Polym Ed 3:229-241.
45. Proudfoot, A. E., C. A. Power, D. J. Church, D. Soler, and M. Mack. 2001. Cellular assays of chemokine receptor activation. Curr Protoc Pharmacol Chapter 12:Unit12 14.
46. Joseph, P. R., K. V. Sawant, A. Isley, M. Pedroza, R. P. Garofalo, R. M. Richardson, and K. Rajarathnam. 2013. Dynamic conformational switching in the chemokine ligand is essential for G Protein coupled-receptor activation. Biochem J.
47. Whittem, C. G., A. D. Williams, and C. S. Williams. 2010. Murine Colitis modeling using Dextran Sulfate Sodium (DSS). J Vis Exp.
48. Perse, M., and A. Cerar. 2012. Dextran sodium sulphate colitis mouse model: traps and tricks. J Biomed Biotechnol 2012:718617.
49. Wirtz, S., C. Neufert, B. Weigmann, and M. F. Neurath. 2007. Chemically induced mouse models of intestinal inflammation. Nat Protoc 2:541-546.
50. Kawada, M., A. Arihiro, and E. Mizoguchi. 2007. Insights from advances in research of chemically induced experimental models of human inflammatory bowel disease. World J Gastroenterol 13:5581-5593.
51. to Velde, A. A., M. I. Verstege, and D. W. Hommes. 2006. Critical appraisal of the current practice in murine TNBS-induced colitis. Inflamm Bowel Dis 12:995-999.
52. Waldner, M. J., and M. F. Neurath. 2009. Chemically induced mouse models of colitis. Curr Protoc Pharmacol Chapter 5:Unit 5 55.
53. Peng, R., S. Sridhar, G. Tyagi, J. E. Phillips, R. Garrido, P. Harris, L. Burns, L. Renteria, J. Woods, L. Chen, J. Allard, P. Ravindran, H. Bitter, Z. Liang, C. M. Hogaboam, C. Kitson, D. C. Budd, J. S. Fine, C. M. Bauer, and C. S. Stevenson. 2013. Bleomycin induces molecular changes directly relevant to idiopathic pulmonary fibrosis: a model for “active” disease. PLoS One 8:e59348.
54. Moeller, A., K. Ask, D. Warburton, J. Gauldie, and M. Kolb. 2008. The bleomycin animal model: a useful tool to investigate treatment options for idiopathic pulmonary fibrosis? Int J Biochem Cell Biol 40:362-382.
55. Mouratis, M. A., and V. Aidinis. 2011. Modeling pulmonary fibrosis with bleomycin. Curr Opin Pulm Med 17:355-361.
56. Walters, D. M., and S. R. Kleeberger. 2008. Mouse models of bleomycin-induced pulmonary fibrosis. Curr Protoc Pharmacol Chapter 5:Unit 5 46.
57. Kawakami, N., I. Bartholomaus, M. Pesic, and M. Mues. 2012. An autoimmunity odyssey: how autoreactive T cells infiltrate into the CNS. Immunol Rev 248:140-155.
58. Attfield, K. E., C. A. Dendrou, and L. Fugger. 2012. Bridging the gap from genetic association to functional understanding: the next generation of mouse models of multiple sclerosis. Immunol Rev 248:10-22.
59. Huang, D. R., J. Wang, P. Kivisakk, B. J. Rollins, and R. M. Ransohoff. 2001. Absence of monocyte chemoattractant protein 1 in mice leads to decreased local macrophage recruitment and antigen-specific T helper cell type 1 immune response in experimental autoimmune encephalomyelitis. J Exp Med 193:713-726.
60. Izikson, L., R. S. Klein, I. F. Charo, H. L. Weiner, and A. D. Luster. 2000. Resistance to experimental autoimmune encephalomyelitis in mice lacking the CC chemokine receptor (CCR)2. J Exp Med 192:1075-1080.
61. Fife, B. T., G. B. Huffnagle, W. A. Kuziel, and W. J. Karpus. 2000. CC chemokine receptor 2 is critical for induction of experimental autoimmune encephalomyelitis. J Exp Med 192:899-905.
62. Rottman, J. B., A. J. Slavin, R. Silva, H. L. Weiner, C. G. Gerard, and W. W. Hancock. 2000. Leukocyte recruitment during onset of experimental allergic encephalomyelitis is CCR1 dependent. Eur J Immunol 30:2372-2377.
63. Karpus, W. J., N. W. Lukacs, B. L. McRae, R. M. Strieter, S. L. Kunkel, and S. D. Miller. 1995. An important role for the chemokine macrophage inflammatory protein-1 alpha in the pathogenesis of the T cell-mediated autoimmune disease, experimental autoimmune encephalomyelitis. J Immunol 155:5003-5010.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

What is claimed is:

1. A method of treating a subject for a disorder that correlates to increased chemokine (C-X-C motif) receptor 8 (CXCR8) signaling, comprising disrupting the activation of receptor CXCR8 by chemokine (C-X-C motif) ligand 17 (CXCL17) in the subject.

2. The method of claim 1, wherein the disrupting comprises administering to the subject a substance that interferes with CXCL17 binding to CXCR8.

3. The method of claim 1, wherein the disorder is a gastrointestinal, respiratory, metabolic, infectious, or oncologic disorder.

4. The method of claim 1, wherein the disorder is a lung, digestive or reproductive system inflammatory disease.

5. The method of claim 4, wherein the inflammatory disease is Crohn's disease (CD), primary sclerosing cholangitis, ulcerative colitis, celiac disease, or irritable bowel syndrome (IBS), an ulcer, ischemic colitis, radiation colitis, celiacs disease, bronchopulmonary dysplasia, idiopathic pulmonary fibrosis, hypersensitivity pneumonitis, non-specific interstitial pneumonia, chronic obstructive pulmonary disease, pneumonia, asthma, bronchitis, emphysema, subclinical interstitial lung disease (subclinical ILD), cystic fibrosis, sarcoidosis, endometriosis, leiomyomas, adenomyosis, bacterial vaginosis, or infections or inflammation of the urethra.

6. The method of claim 1, wherein the substance is an antibody that binds to CXCL17 or CXCR8, a polypeptide sequence variant of CXCL17, a non-peptide conjugation variant of CXCL17, a small molecule that binds to CXCL17 or CXCR8, or an aptamer that binds to CXCL17 or CXCR8.

7. The method of claim 1, wherein the disorder is a gastrointestinal, respiratory, metabolic, infectious, or oncologic disorder, and the substance is an antagonist of CXCL17.

8. The method of claim 7, wherein said antagonist is selected from:
a) an antibody, or a fragment thereof, which binds to CXCR8;
b) a CXCL17 variant; or
c) a small molecule compound.

9. The method of claim 7, wherein said gastrointestinal disorder which correlates to increased CXCR8 signaling is selected from the group consisting of:
a) Crohn's disease (CD), ulcerative colitis, celiac disease, or irritable bowel syndrome (IBS), ischemic colitis, radiation colitis, celiacs disease;
b) stomach cancer, pancreatic cancer, colorectal cancer, or hepatocellular carcinoma, esophageal cancer, liver cancer, gallbladder cancer, biliary cancer, gastrointestinal stromal tumors;
c) autoimmune hepatitis, primary biliary cirrhosis, other (non autoimmune) cirrhosis, primary sclerosing cholangitis, liver fibrosis; and
d) hepatitis C virus (HCV) mediated cirrhosis, and peptic ulcers caused by Helicobacter pylori.

10. The method of claim 7, wherein said metabolic disorder which correlates to increased CXCR8 signaling is diabetes type 1, or diabetes type 2.

11. The method of claim 7, wherein said oncology disorder is a leukemia or a lymphoma.

12. The method of claim 11, wherein said leukemia or lymphoma expresses CXCR8.

13. The method of claim 7, wherein said oncology disorder is glioblastoma or related brain tumor.

14. The method of claim 7, wherein said respiratory system disorder which correlates to increased CXCR8 signaling is selected from the group consisting of:
a) lung cancer, including small or non-small cell lung cancer or mesothelioma (malignant);
b) idiopathic pulmonary fibrosis, hypersensitivity pneumonitis, or non-specific interstitial pneumonia;
c) a respiratory disease associated with interstitial lung disorders including autoimmune diseases like rheumatoid arthritis or scleroderma;
d) chronic obstructive pulmonary disease (COPD), bronchopulmonary dysplasia (BPD), asthma; and
e) another respiratory cancer.

15. The method of claim 14, wherein the another respiratory cancer is trachea cancer, cancer of the larynx, cancer of the bronchus, or nasal/sinus cancer.

16. The method of claim 14, wherein said administering is:
a) topical, local, or systemic;
b) inhaled as an aerosol or mist; or
c) in combination with another therapeutic.

17-19. (canceled)

20. A method of treating or preventing atherosclerosis, said method comprising administering an effective amount of:
a) a CXCR8 modulator; or a modulator of CXCR8 expression; or
b) a CXCL17 modulator; or a modulator of CXCL17 expression.

21. The method of claim 20, wherein said CXCR8 modulator is selected from the group consisting of:
a) an antibody that binds to CXCR8 or CXCR8 variant;
b) a polypeptide sequence variant of CXCL17;
c) a non-peptide conjugation variant of CXCL17;
d) a small molecule antagonist candidate; and
e) an aptamer.

22. (canceled)

23. The method of claim 20, wherein said CXCL17 modulator is selected from the group consisting of:
a) an antibody that binds to CXCL17 or CXCL17 variant;
b) a polypeptide sequence variant of CXCL17;
c) a non-peptide conjugation variant of CXCL17;
d) a small molecule antagonist; and
e) an aptamer.

24-26. (canceled)

27. A method that identifies CXCR8 as a marker of cells involved in the pathogenesis of human disease, wherein the disease is gastrointestinal, metabolic or respiratory disease, or cancer.

28. The method of claim 27, wherein said CXCR8 is a biomarker of metastatic cells of leukemias, lymphomas, stomach cancer, colorectal cancer or pancreatic cancer, or is a biomarker of subclinical interstitial lung disease (subclinical ILD).

29. The method of claim 27, wherein said CXCR8 is a biomarker of metastatic cells of lung cancer including small or non-small cell lung cancer or malignant mesothelioma.

30. The method of claim 27, wherein said CXCR8 is a prognostic biomarker of cells that infiltrate gastrointestinal or respiratory system cancers.

31-34. (canceled)

35. A method of modulating CXCL17 signaling through CXCR8, said method comprising contacting:
a) CXCR8 with a CXCL17 modulator;
b) CXCL17 with a blocking agent; or
c) the cell expressing CXCR8 with a modulator of cell signaling.

36. The method of claim 35, wherein said CXCL17 modulator is selected from the group consisting of:
a) an antibody, or a fragment thereof, which binds to CXCR8 or CXCR8 variant;
b) a CXCL17 variant; and
c) a small molecule compound.

37. The method of claim 35, wherein said blocking agent is selected from the group consisting of:
a) an antibody, or a fragment thereof, which binds to CXCL17 or CXCL17 variant;
b) a fragment of the CXCR8 receptor; and
c) a small molecule compound.

38. The method of claim 35, wherein said modulator of cell signaling is:
a) an RNAi, CRISPR, or TALEN compound of signaling pathway members;
b) an antibody which blocks signaling pathway; or
c) a small molecule blocker of signaling pathway.

39. A method of screening for said CXCL17 modulator of claim 35, wherein said screening comprises a cell based assay comprising a fluorescent imaging plate reader (FLIPR) or related detection.

40. The method of claim 39, wherein said screening is of one or more compounds which include:
a) antibodies binding to CXCL17 or CXCL17 variant;
b) polypeptide sequence variants of CXCL17;
c) non-peptide conjugation variants of CXCL17;
d) small molecule modulator candidates; or
e) aptamer libraries.

41. A method of screening for said blocking agent of claim 35, wherein said screening comprises an assay comprising a fluorescent imaging plate reader (FLIPR) or related detection.

42. The method of claim 41, wherein said screening is of one or more compounds which include:
a) antibodies binding to CXCR8 or CXCR8 variant;
b) polypeptide sequence variants of CXCL17;
c) non-peptide conjugation variants of CXCL17;
d) small molecule antagonist candidates; or
e) aptamer libraries.

43. The method of claim 41, wherein CXCR8 transfectants of cell line Ba/F3 are used to screen for agonists and antagonists of CXCR8/CXCL17 interaction.

44. (canceled)

45. A method of inhibiting CXCL17 signaling through CXCR8, said method comprising reducing CXCL17 using an RNAi, CRISPR, or TALEN compound which inhibits expression of CXCL17.

46. A method of inducing CXCR8 signaling, said method comprising contacting said receptor with its cognate ligand.

47. The method of claim 46, wherein said cognate ligand is CXCL17 or an agonist thereof.

48. The method of claim 47, wherein said agonist is a polypeptide sequence variant of CXCL17 or a non-peptide conjugation variant of CXCL17.

49-52. (canceled)

53. A ligand of CXCR8, wherein said ligand binds selectively to the CXCR8 receptor.

54. The ligand of claim 53, wherein said ligand:
a) signals through said receptor;
b) signals less than 90% of human CXCL17;
c) is an inverse agonist of CXCR8;
d) is an allosteric modulator of CXCR8;
e) is a polypeptide sequence variant of human CXCL17;
f) comprises a segment of at least 17 amino acids exhibiting at least 97% identity to human CXCL17; or
g) binds to primate CXCR8 receptor.

55. The ligand of claim 53, wherein said ligand:
a) is in a sterile composition;
b) is formulated for systemic administration;
c) is in a therapeutic composition;
d) is in a single dose container; or
e) is a polypeptide sequence variant of human CXCL17.

56. An antibody which binds selectively to the ligand of claim 53 and:
a) blocks binding to said CXCR8 receptor; or
b) blocks signaling by the CXCR8 receptor.

57. A receptor for human CXCL17, wherein said receptor is CXCR8.

58. The receptor of claim 57, wherein said receptor:
a) further signals upon binding of said human CXCL17;
b) signals at least 80% of signal upon binding of CXCL17 compared to human CXCR8;
c) has at least 95% identity to human CXCR8; or
d) binds to primate CXCL17.

59-64. (canceled)

65. A method of treating or preventing multiple sclerosis, said method comprising administering an effective amount of:
a) a CXCR8 modulator; or a modulator of CXCR8 expression; or
b) a CXCL17 modulator; or a modulator of CXCL17 expression.

66. The method of claim 65, wherein said CXCR8 modulator is selected from the group consisting of:
a) an antibody that binds to CXCR8 or CXCR8 variant;
b) a polypeptide sequence variant of CXCL17;
c) a non-peptide conjugation variant of CXCL17;
d) a small molecule antagonist candidate; and
e) an aptamer.

67. The method of claim 65, wherein said CXCL17 modulator is selected from the group consisting of:
a) an antibody that binds to CXCL17 or CXCL17 variant;
b) a polypeptide sequence variant of CXCL17;
c) a non-peptide conjugation variant of CXCL17;
d) a small molecule antagonist; and
e) an aptamer.
